(19) United States
(12) Patent Application Publication
(10) Pub. No.: US 2025/0230243 A1
(43) Pub. Date: Jul. 17, 2025

(54) PREDICTION OF CHEMOTHERAPY-FREE IMMUNOTHERAPY EFFECTIVENESS FOR PATIENTS WITH CANCERS

(71) Applicant: Foundation Medicine, Inc., Boston, MA (US)

(72) Inventor: Ryon P. GRAF, Encinitas, CA (US)

(73) Assignee: Foundation Medicine, Inc., Boston, MA (US)

(21) Appl. No.: 19/017,134

(22) Filed: Jan. 10, 2025

Related U.S. Application Data
(60) Provisional application No. 63/620,099, filed on Jan. 11, 2024.

(51) Int. Cl. C07K 16/28; C12Q 1/6886

(52) U.S. Cl. CPC C07K 16/2818; C07K 16/2827; C12Q 1/6886; C07K 2317/24; C12Q 2600/106; C12Q 2600/156

(57) ABSTRACT

Disclosed herein are methods of treating an individual having a gastroesophageal cancer, of treating or identifying an individual having gastroesophageal cancer for a treatment, or stratifying individuals having a gastroesophageal cancer for a treatment based on a tumor mutational burden (TMB) score or a TMB score.

### CROSS-REFERENCE TO RELATED APPLICATIONS

This application claims priority to U.S. Provisional Patent Application No. 63/620,099, filed Jan. 11, 2024, which is incorporated herein by reference in its entirety.

### FIELD

Provided herein are methods of selecting a treatment for an individual having a gastroesophageal cancer, of treating or identifying an individual having a gastroesophageal cancer for a treatment, or stratifying individuals having gastroesophageal cancer for a treatment based on a tumor mutational burden (TMB) score.

### BACKGROUND

Cancer can be caused by genomic mutations, and cancer cells may accumulate mutations during cancer development and progression. These mutations may be the consequence of intrinsic malfunction of DNA repair, replication, or modification mechanisms, or may be a consequence of exposure to external mutagens. Certain mutations confer growth advantages on cancer cells and are positively selected in the microenvironment of the tissue in which the cancer arises. Detection of these mutations in patient samples using next generation sequencing (NGS) or other genomic analysis techniques can provide valuable insights with respect to diagnosis, prognosis, and treatment of cancer.

Immune checkpoint inhibitor (ICPI) therapy combined with chemotherapy is becoming an increasingly common first line regimen for patients with advanced gastroesophageal cancer. However, direct outcome comparison between biomarker-enriched groups receiving ICPI and chemotherapy versus ICPI monotherapy is limited in phase III clinical trials. Accordingly, there is a need to identify groups of patients with gastroesophageal cancer who have comparable or superior outcomes on ICPI monotherapy compared to ICPI with chemotherapy in a first line regimen.

### BRIEF SUMMARY OF THE INVENTION

In some aspects, provided herein is a method for treating an individual having a gastroesophageal cancer, the method comprising: (a) determining a tumor mutational burden (TMB) score for a biopsy sample obtained from the individual; and (b) administering an immune checkpoint inhibitor without a chemotherapy regimen to the individual if the TMB score is at or higher than a threshold TMB score.

In some aspects, provided herein is a method for identifying an individual having a gastroesophageal cancer for treatment with an immune checkpoint inhibitor therapy without a chemotherapy regimen comprising determining a tumor mutational burden (TMB) score for a biopsy sample obtained from the individual, wherein if the TMB score is at or higher than a threshold TMB score the individual is identified for treatment with an immune checkpoint inhibitor therapy without a chemotherapy regimen.

In some aspects, provided herein is a method of selecting a treatment for an individual having a gastroesophageal cancer, the method comprising determining a tumor mutational burden (TMB) score for a biopsy sample obtained from the individual, wherein a TMB score that is at or higher than a threshold TMB score identifies the individual as one who may benefit from treatment with an immune checkpoint inhibitor therapy without a chemotherapy regimen.

In some aspects, provided herein is a method of identifying one or more treatment options for an individual having a gastroesophageal cancer, the method comprising: (a) determining a tumor mutational burden (TMB) score for a biopsy sample obtained from the individual; and (b) generating a report comprising one or more treatment options identified for the individual, wherein a TMB score that is at or higher than a threshold TMB score identifies the individual as one who may benefit from treatment with an immune checkpoint inhibitor therapy without a chemotherapy regimen.

In some aspects, provided herein is a method of stratifying an individual with a gastroesophageal cancer for treatment with a therapy comprising determining a tumor mutational burden (TMB) score for a biopsy sample obtained from the individual; and (a) if the TMB score is at or higher than a threshold TMB score, identifying the individual as a candidate for receiving an immune checkpoint inhibitor therapy without a chemotherapy regimen, or (b) if the TMB score is less than a threshold TMB score, identifying the individual as a candidate for receiving an immune checkpoint inhibitor therapy and a chemotherapy regimen.

In some aspects, provided herein is a method of predicting survival of an individual having a gastroesophageal cancer, comprising acquiring knowledge of a tumor mutational burden (TMB) score for a biopsy sample obtained from the individual, wherein if the TMB score for the biopsy sample obtained from the individual is at or higher than a threshold TMB score, the individual is predicted to have increased survival when treated with an immune checkpoint inhibitor without a chemotherapy regimen, as compared to treatment with an immune checkpoint inhibitor therapy and a chemotherapy regimen.

In some aspects, provided herein is a method of monitoring, evaluating, or screening an individual having a gastroesophageal cancer, comprising acquiring knowledge of a tumor mutational burden (TMB) score for a biopsy sample obtained from the individual, wherein if the TMB score for the biopsy sample obtained from the individual is at or higher than a threshold TMB score, the individual is predicted to have increased survival when treated with an immune checkpoint inhibitor without a chemotherapy regimen, as compared to treatment with an immune checkpoint inhibitor therapy and a chemotherapy regimen.

In some embodiments, the threshold TMB score is about 8 mutations/Mb, about 9 mutations/Mb, about 10 mutations/Mb, about 11 mutations/Mb, about 12 mutations/Mb, about 13 mutations/Mb, about 14 mutations/Mb, about 15 mutations/Mb, about 16 mutations/Mb, about 17 mutations/Mb, about 18 mutations/Mb, about 19 mutations/Mb, or about 20 mutations/Mb. In some embodiments, the threshold TMB score is about 10 mutations/Mb.

In some embodiments, the TMB score is determined based on between about 100 kb to about 10 Mb of sequenced DNA. In some embodiments, the TMB score is determined based on between about 0.8 Mb to about 1.1 Mb of sequenced DNA.

In some embodiments, the method further comprises administering an immune checkpoint inhibitor without a chemotherapy regimen to the individual if the TMB score is at or higher than the threshold TMB score.

In some embodiments, the method further comprises administering an immune checkpoint inhibitor and a chemotherapy regimen to the individual if the TMB score is less than the threshold TMB score.

In some embodiments, the immune checkpoint inhibitor therapy is a first line therapy for the gastroesophageal cancer.

In some embodiments, the immune checkpoint inhibitor comprises a small molecule inhibitor, an antibody, a nucleic acid, an antibody-drug conjugate, a recombinant protein, a fusion protein, a natural compound, a peptide, a PROteolysis-TArgeting Chimera (PROTAC), a cellular therapy, a treatment for cancer being tested in a clinical trial, an immunotherapy, or any combination thereof.

In some embodiments, the immune checkpoint inhibitor is a PD-1 inhibitor. In some embodiments, the PD-1 inhibitor comprises one or more of nivolumab, pembrolizumab, cemiplimab, or dostarlimab.

In some embodiments, the immune checkpoint inhibitor is a PD-L1 inhibitor. In some embodiments, the PD-L1 inhibitor comprises one or more of atezolizumab, avelumab, or durvalumab.

In some embodiments, the immune checkpoint inhibitor is a CTLA-4 inhibitor. In some embodiments, the CTLA-4 inhibitor comprises ipilimumab.

In some embodiments, the immune checkpoint inhibitor therapy is the only anti-cancer therapy indicated or administered for the gastroesophageal cancer. In some embodiments, the immune checkpoint inhibitor therapy is a single-active-agent therapy. In some embodiments, the immune checkpoint inhibitor therapy comprises two or more active agents.

In some embodiments, the immune checkpoint inhibitor therapy comprises a first round of an immune checkpoint inhibitor and a subsequent round of therapy with a different immune checkpoint inhibitor.

In some embodiments, the chemotherapy comprises one or more of an alkylating agent, an alkyl sulfonates aziridine, an ethylenimine, a methylamelamine, an acetogenin, a camptothecin, a bryostatin, a callystatin, CC-1065, a cryptophycin, aa dolastatin, a duocarmycin, a eleutherobin, a pancratistatin, a sarcodictyin, a spongistatin, a nitrogen mustard, a nitrosureas, an antibiotic, a dynemicin, a bisphosphonate, an esperamicina a neocarzinostatin chromophore or a related chromoprotein enediyne antiobiotic chromophore, an anti-metabolite, a folic acid analogue, a purine analog, a pyrimidine analog, an androgens, an anti-adrenal, a folic acid replenisher, aldophosphamide glycoside, aminolevulinic acid, eniluracil, amsacrine, bestrabucil, bisantrene, edatraxate, defofamine, demecolcine, diaziquone, elformithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, a PSK polysaccharide complex, razoxane, rhizoxin, sizofiran, spirogermanium, tenuazonic acid, triaziquone, 2,2′,2″-trichlorotriethylamine, a trichothecene, urethan, vindesine, dacarbazine, mannomustine, mitobronitol, mitolactol, pipobroman, gacytosine, arabinoside (“Ara-C”), cyclophosphamide, a taxoid, 6-thioguanine, mercaptopurine, a platinum coordination complex, vinblastine, platinum, etoposide (VP-16), ifosfamide, mitoxantrone, vincristine, vinorelbine, novantrone, teniposide, edatrexate, daunomycin, aminopterin, xeloda, ibandronate, irinotecan, topoisomerase inhibitor RFS 2000, difluorometlhylomithine (DMFO), a retinoid, Capecitabine, carboplatin, procarbazine, plicomycin, gemcitabine, navelbine, a famesyl-protein tansferase inhibitor, transplatinum, fluorouracil, a taxane, or any combination thereof.

In some embodiments, the method further comprises administering an additional anti-cancer therapy to the individual. In some embodiments, the additional anti-cancer therapy comprises one or more of a small molecule inhibitor, a cancer immunotherapy, an antibody, a cellular therapy, a nucleic acid, a surgery, a radiotherapy, an anti-angiogenic therapy, an anti-DNA repair therapy, an anti-inflammatory therapy, a growth inhibitory agent, or any combination thereof.

In some embodiments, the individual did not previously receive a regimen of chemotherapy for the gastroesophageal cancer.

In some embodiments, the TMB score is determined by sequencing. In some embodiments, the sequencing comprises use of a massively parallel sequencing (MPS) technique, whole genome sequencing (WGS), whole exome sequencing (WES), targeted sequencing, direct sequencing, next-generation sequencing (NGS), or a Sanger sequencing technique. In some embodiments, the sequencing comprises: (a) providing a plurality of nucleic acid molecules obtained from the biopsy sample, wherein the plurality of nucleic acid molecules comprise a mixture of tumor nucleic acid molecules and non-tumor nucleic acid molecules; (b) optionally, ligating one or more adapters onto one or more nucleic acid molecules from the plurality of nucleic acid molecules; (c) amplifying nucleic acid molecules from the plurality of nucleic acid molecules; (d) capturing nucleic acid molecules from the amplified nucleic acid molecules, wherein the captured nucleic acid molecules are captured from the amplified nucleic acid molecules by hybridization to one or more bait molecules; and (e) sequencing, by a sequencer, at or higher than a portion of the captured nucleic acid molecules to obtain a plurality of sequence reads corresponding to one or more genomic loci within a subgenomic interval in the sample. In some embodiments, the adapters comprise one or more of amplification primer sequences, flow cell adapter hybridization sequences, unique molecular identifier sequences, substrate adapter sequences, or sample index sequences. In some embodiments, amplyifying nucleic acid molecules comprises performing a polymerase chain reaction (PCR) technique, a non-PCR amplification technique, or an isothermal amplification technique. In some embodiments, the one or more bait molecules comprise one or more nucleic acid molecules, each comprising a region that is complementary to a region of a captured nucleic acid molecule. In some embodiments, the one or more bait molecules each comprise a capture moiety. In some embodiments, the capture moiety is biotin.

In some embodiments, if the TMB score is at or higher than a threshold TMB score, the individual is identified to have an increased survival when treated with an immune checkpoint inhibitor without a chemotherapy regimen as compared to treatment with an immune checkpoint inhibitor therapy and a chemotherapy regimen. In some embodiments, the increased survival is increased overall survival (OS). In some embodiments, the increased survival is increased progression-free survival (PFS).

In some embodiments, the gastroesophageal cancer is advanced. In some embodiments the gastroesophageal cancer is metastatic.

In some embodiments, the biopsy sample is a tumor biopsy sample. In some embodiments, the biopsy sample is a liquid biopsy sample.

In some embodiments, the individual is a human.

In some aspects, provided herein in a kit comprising an immune checkpoint inhibitor and instructions for use according to any one of the methods provided herein.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples, and are incorporated in and constitute a part of this specification, but are not intended as a definition of the limits of a particular example. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure.

FIGS. 1A-1B show the cohort selection diagram (FIG. 1A) and analysis overview (FIG. 1B). Acronyms: CTLA-4: cytotoxic T-lymphocyte-associated protein 4; Dx: diagnosis; HER2: human epidermal growth factor receptor 2; ICI: immune checkpoint inhibitor; MSI: microsatellite instability; PD1: programmed death 1; Q2: second quarter; rwOS: real-world overall survival; rwPFS: real-world progression-free survival; TMB: tumor mutational burden; Tx: treatment; and PFS: progression-free survival.

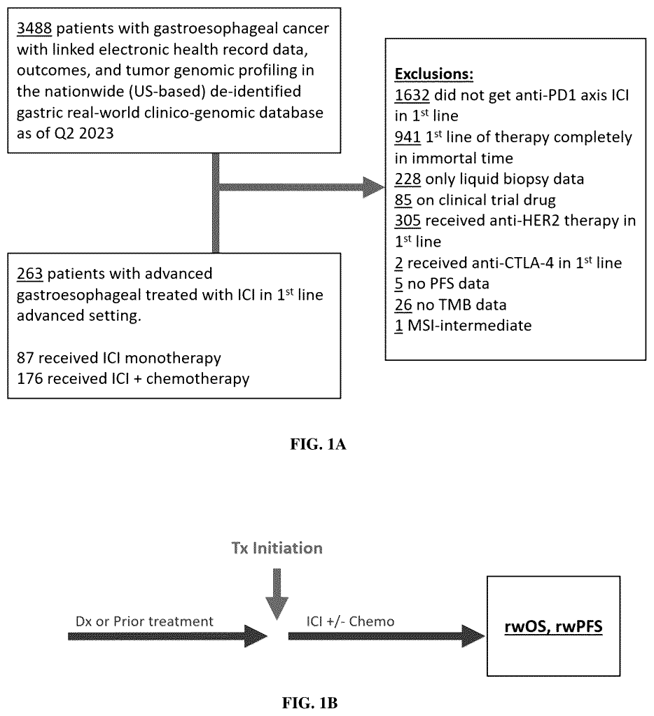

FIGS. 2A-2D demonstrate the addition of chemotherapy to ICI is most effective when TMB<10. Presented are Kaplan-Meier curves adjusted for treatment assignment imbalances. rwPFS is shown by drug class (single-agent ICI=*; ICI with chemotherapy=\hat{ }) for TMB<10 (FIG. 2A) and TMB≥10 (FIG. 2B). rwOS is shown by drug class for TMB<10 (FIG. 2C) and TMB≥10 (FIG. 2D). Overall survival estimates are left truncated to reflect delayed entry to at-risk table. Visualizations are adjusted by propensity weights. Analyses unadjusted for propensity weights have similar results (FIGS. 4A-4D). Interaction terms in interaction models for TTNT and OS respectively p=0.0056 and p=0.013 (for full models, see FIGS. 4A-4D). Acronyms: HR: hazard ratio; OS: overall survival; rwOS: real-world overall survival; rwPFS: real-world progression-free survival; TTNT: time to next treatment; TMB: tumor mutational burden; Tx: treatment; and 1 L: 1st line.

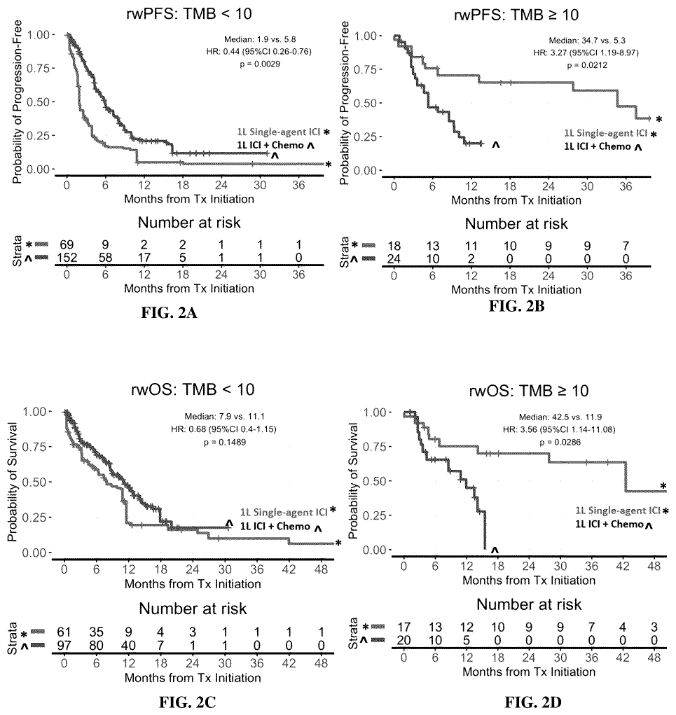

FIG. 3 demonstrates TMB is more predictive than MSI for 1st line ICI monotherapy rwPFS. Presented is a waterfall plot showing rwPFS associated with all patients treated with 1st line ICI monotherapy. Acronyms: ICI: immune checkpoint inhibitor; rwPFS: real-world progression-free survival; rwOS: real-world overall survival; MSI-H: microsatellite instability high; MSS: microsatellite stable; and TTD: time to treatment discontinuation.

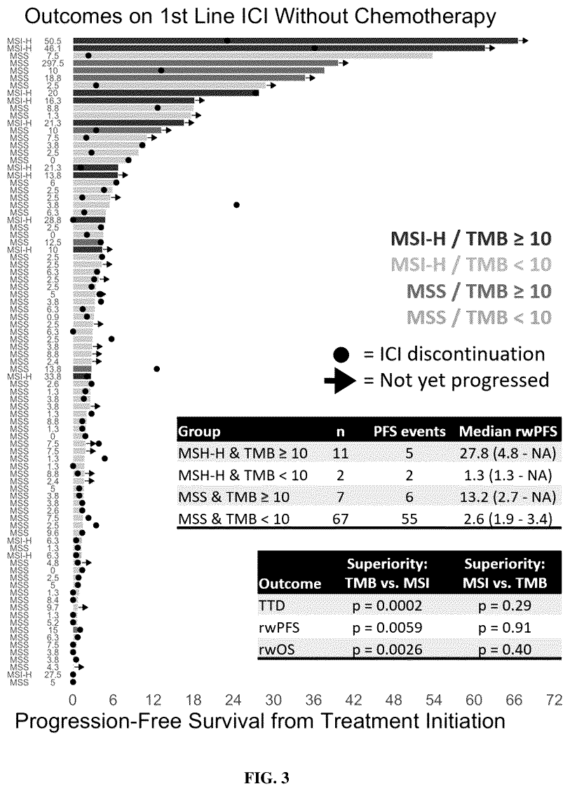

FIGS. 4A-4D demonstrate the addition of chemotherapy to ICI is most effective when TMB<10. Presented are Kaplan-Meier curves unadjusted for treatment assignment imbalances. rwPFS is shown by drug class (single-agent ICI=*; ICI with chemotherapy=\hat{ }) for TMB<10 (FIG. 4A) and TMB≥10 (FIG. 4B). rwOS is shown by drug class for TMB<10 (FIG. 4C) and TMB≥10 (FIG. 4D). Overall survival estimates are left truncated to reflect delayed entry to at-risk table.

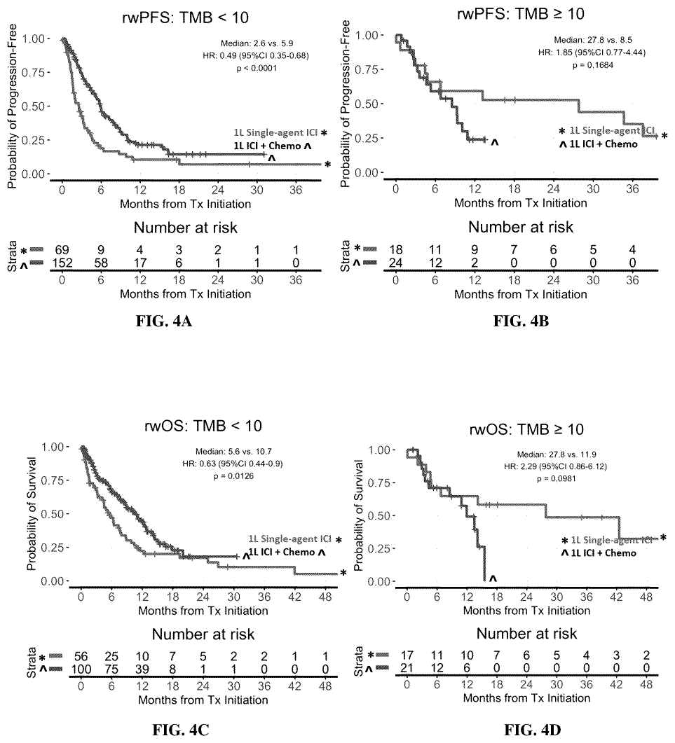

FIGS. 5A-5B show the post-propensity weighted balance of factors known to affect treatment assignment of ICI or ICI+chemotherapy. Dashed lines indicate 10% standard mean difference, a level considered acceptable for balance adjustment by standard statistical practice in causal inference. Acronyms: StatageIV.numeric=Whether patient was stage IV at diagnosis or nor (yes vs. no); PDL1.unknown=whether PD-L1 staining status was unknown (yes vs. no); PDL1.CPS10: whether PD-L1 staining had a combined combined score of 10% or greater (yes vs. no); MSI-h: whether patient was microsatellite instability high (yes vs. no); ECOG2: whether patient was ECOG score of 2 or greater (yes vs. no); ECOG1: whether patient was ECOG score of 1 or other (yes vs. no); ECOG0: whether patient was ECOG score of 0 or other (yes vs. no); and ECOG=Eastern Cooperative Oncology Group.

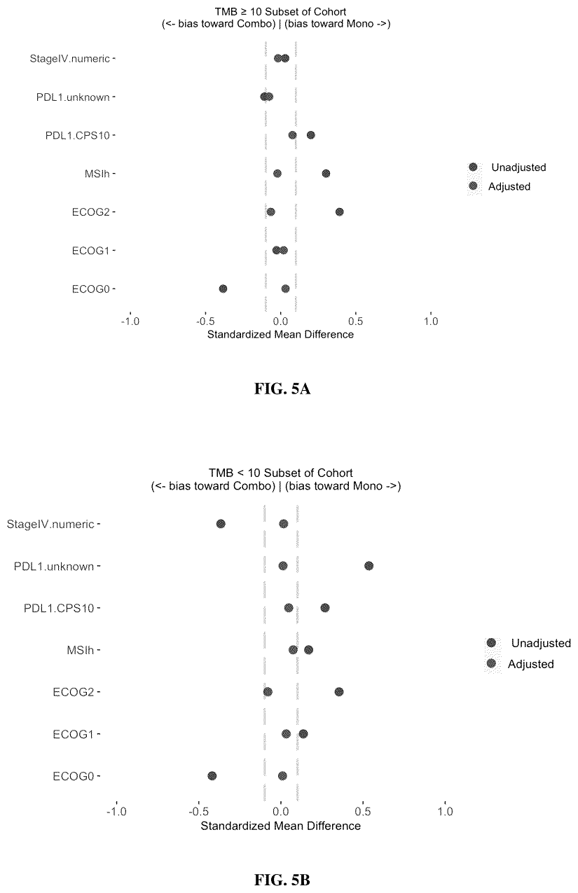

### DETAILED DESCRIPTION

Described herein are methods comprising determining a tumor mutational burden (TMB) score for a sample obtained from an individual having a gastroesophageal cancer and comparing the determined TMB score with a threshold TMB score. It has been discovered that comparison of the determined TMB score with a threshold TMB score can help guide treatment decisions, including selecting between an immune checkpoint inhibitor (ICPI) therapy without a chemotherapy regimen and an ICPI with a chemotherapy regimen.

Thus, described herein are methods for treating an individual having a gastroesophageal cancer, the method comprising: (a) determining a tumor mutational burden (TMB) score for a biopsy sample obtained from the individual; and (b) administering an immune checkpoint inhibitor without a chemotherapy regimen to the individual if the TMB score is at or higher than a threshold TMB score. Further described herein are methods of identifying an individual having a gastroesophageal cancer for treatment with an immune checkpoint inhibitor therapy comprising determining a tumor mutational burden (TMB) score for a biopsy sample obtained from the individual, wherein if the TMB score is at or higher than a threshold TMB score the individual is identified for treatment with an immune checkpoint inhibitor therapy without a chemotherapy regimen. Further described herein are methods of selecting a treatment for an individual having a gastroesophageal cancer, the method comprising determining a tumor mutational burden (TMB) score for a biopsy sample obtained from the individual, wherein a TMB score that is at or higher than a threshold TMB score identifies the individual as one who may benefit from treatment with an immune checkpoint inhibitor therapy without a chemotherapy regimen. Further described herein are methods of identifying one or more treatment options for an individual having a gastroesophageal cancer, the method comprising: (a) determining a tumor mutational burden (TMB) score for a biopsy sample obtained from the individual; and (b) generating a report comprising one or more treatment options identified for the individual, wherein a TMB score that is at or higher than a threshold TMB score identifies the individual as one who may benefit from treatment with an immune checkpoint inhibitor therapy without a chemotherapy regimen. Further described herein are methods of stratifying an individual with a gastroesophageal cancer for treatment with a therapy comprising determining a tumor mutational burden (TMB) score for a biopsy sample obtained from the individual; and (a) if the TMB score is at or higher than a threshold TMB score, identifying the individual as a candidate for receiving an immune checkpoint inhibitor therapy without a chemotherapy regimen, or (b) if the TMB score is less than a threshold TMB score, identifying the individual as a candidate for receiving an immune checkpoint inhibitor therapy and a chemotherapy regimen. Further described herein are methods of predicting survival of an individual having a gastroesophageal cancer, comprising acquiring knowledge of a tumor mutational burden (TMB) score for a biopsy sample obtained from the individual, wherein if the TMB score for the biopsy sample obtained from the individual is at or higher than a threshold TMB score, the individual is predicted to have increased survival when treated with an immune checkpoint inhibitor without a chemotherapy regimen, as compared to treatment with an immune checkpoint inhibitor therapy and a chemotherapy regimen. Further described herein are methods of monitoring, evaluating, or screening an individual having a gastroesophageal cancer, comprising acquiring knowledge of a tumor mutational burden (TMB) score for a biopsy sample obtained from the individual, wherein if the TMB score for the biopsy sample obtained from the individual is at or higher than a threshold TMB score, the individual is predicted to have increased survival when treated with an immune checkpoint inhibitor without a chemotherapy regimen, as compared to treatment with an immune checkpoint inhibitor therapy and a chemotherapy regimen. In some embodiments of these methods, the sample is a tumor biopsy sample. In some embodiments of these methods, the sample is a blood sample.

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. De Vita et al., eds., J. B. Lippincott Company, 1993).

### II. Definitions

Certain terms are defined. Additional terms are defined throughout the specification.

As used herein, the articles “a” and “an” refer to one or to more than one (e.g., to at least one) of the grammatical object of the article.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

It is understood that aspects and embodiments of the invention described herein include “comprising,” “consisting,” and “consisting essentially of” aspects and embodiments.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

“Polynucleotide,” “nucleic acid,” or “nucleic acid molecule” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-0-methyl-, 2′-0-allyl-, 2′-fluoro-, or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(0)S (“thioate”), P(S)S (“dithioate”), “(0) NR₂ (“amidate”), P(0)R, P(0)OR′, CO or CH₂ (“formacetal”), in which each R or R′ is independently H or substituted or unsubstituted alkyl (1-20 C) optionally containing an ether (-0-) linkage, aryl, alkenyl, cycloalkyl, cycloalkenyl or araldyl. Not all linkages in a polynucleotide need be identical. A polynucleotide can contain one or more different types of modifications as described herein and/or multiple modifications of the same type. The preceding description applies to all polynucleotides referred to herein, including RNA and DNA.

The term “detection” includes any means of detecting, including direct and indirect detection. The term “biomarker” as used herein refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features (e.g., responsiveness to therapy, e.g., a checkpoint inhibitor). In some embodiments, a biomarker is a collection of genes or a collective number of mutations/alterations (e.g., somatic mutations) in a collection of genes. Biomarkers include, but are not limited to, polynucleotides (e.g., DNA and/or RNA), polynucleotide alterations (e.g., polynucleotide copy number alterations, e.g., DNA copy number alterations, or other mutations or alterations), polypeptides, polypeptide and polynucleotide modifications (e.g., post-translational modifications), carbohydrates, and/or glycolipid-based molecular markers.

“Amplification,” as used herein generally refers to the process of producing multiple copies of a desired sequence. “Multiple copies” means at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

“Individual response” or “response” can be assessed using any endpoint indicating a benefit to the individual, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g., cancer); (6) increase or extension in the length of survival, including overall survival and progression free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and/or progression-free survival); resulting in an objective response (including a complete response or a partial response); or improving signs or symptoms of cancer.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the patient herein is a human.

As used herein, “administering” is meant a method of giving a dosage of an agent or a pharmaceutical composition (e.g., a pharmaceutical composition including the agent) to a subject (e.g., a patient). Administering can be by any suitable means, including parenteral, intrapulmonary, and intranasal, and, if desired for local treatment, intralesional administration. Parenteral infusions include, for example, intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The terms “concurrently” or “in combination” are used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

“Acquire” or “acquiring” as the terms are used herein, refer to obtaining possession of a physical entity, or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third-party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

“Acquiring a sequence” or “acquiring a read” as the term is used herein, refers to obtaining possession of a nucleotide sequence or amino acid sequence, by “directly acquiring” or “indirectly acquiring” the sequence or read. “Directly acquiring” a sequence or read means performing a process (e.g., performing a synthetic or analytical method) to obtain the sequence, such as performing a sequencing method (e.g., a Next-generation Sequencing (NGS) method). “Indirectly acquiring” a sequence or read refers to receiving information or knowledge of, or receiving, the sequence from another party or source (e.g., a third-party laboratory that directly acquired the sequence). The sequence or read acquired need not be a full sequence, e.g., sequencing of at least one nucleotide, or obtaining information or knowledge, that identifies one or more of the alterations disclosed herein as being present in a sample, biopsy or subject constitutes acquiring a sequence.

Directly acquiring a sequence or read includes performing a process that includes a physical change in a physical substance, e.g., a starting material, such as a sample described herein. Exemplary changes include making a physical entity from two or more starting materials, shearing or fragmenting a substance, such as a genomic DNA fragment; separating or purifying a substance (e.g., isolating a nucleic acid sample from a tissue); combining two or more separate entities into a mixture, performing a chemical reaction that includes breaking or forming a covalent or non-covalent bond. Directly acquiring a value includes performing a process that includes a physical change in a sample or another substance as described above. The size of the fragment (e.g., the average size of the fragments) can be 2500 bp or less, 2000 bp or less, 1500 bp or less, 1000 bp or less, 800 bp or less, 600 bp or less, 400 bp or less, or 200 bp or less. In some embodiments, the size of the fragment (e.g., cfDNA) is between about 150 bp and about 200 bp (e.g., between about 160 bp and about 170 bp). In some embodiments, the size of the fragment (e.g., DNA fragments from liquid biopsy samples) is between about 150 bp and about 250 bp. In some embodiments, the size of the fragment (e.g., cDNA fragments obtained from RNA in liquid biopsy samples) is between about 100 bp and about 150 bp.

“Alteration” or “altered structure” as used herein, of a gene or gene product (e.g., a marker gene or gene product) refers to the presence of a mutation or mutations within the gene or gene product, e.g., a mutation, which affects integrity, sequence, structure, amount or activity of the gene or gene product, as compared to the normal or wild-type gene. The alteration can be in amount, structure, and/or activity in a cancer tissue or cancer cell, as compared to its amount, structure, and/or activity, in a normal or healthy tissue or cell (e.g., a control), and is associated with a disease state, such as cancer. For example, an alteration which is associated with cancer, or predictive of responsiveness to anti-cancer therapeutics, can have an altered nucleotide sequence (e.g., a mutation), amino acid sequence, chromosomal translocation, intra-chromosomal inversion, copy number, expression level, protein level, protein activity, epigenetic modification (e.g., methylation or acetylation status, or post-translational modification, in a cancer tissue or cancer cell, as compared to a normal, healthy tissue or cell. Exemplary mutations include, but are not limited to, point mutations (e.g., silent, missense, or nonsense), deletions, insertions, inversions, duplications, amplification, translocations, inter- and intra-chromosomal rearrangements. Mutations can be present in the coding or non-coding region of the gene. In certain embodiments, the alteration(s) is detected as a rearrangement, e.g., a genomic rearrangement comprising one or more introns or fragments thereof (e.g., one or more rearrangements in the 5′- and/or 3′-UTR). In certain embodiments, the alterations are associated (or not associated) with a phenotype, e.g., a cancerous phenotype (e.g., one or more of cancer risk, cancer progression, cancer treatment or resistance to cancer treatment). In one embodiment, the alteration (or tumor mutational burden) is associated with one or more of: a genetic risk factor for cancer, a positive treatment response predictor, a negative treatment response predictor, a positive prognostic factor, a negative prognostic factor, or a diagnostic factor.

As used herein, the term “indel” refers to an insertion, a deletion, or both, of one or more nucleotides in a nucleic acid of a cell. In certain embodiments, an indel includes both an insertion and a deletion of one or more nucleotides, where both the insertion and the deletion are nearby on the nucleic acid. In certain embodiments, the indel results in a net change in the total number of nucleotides. In certain embodiments, the indel results in a net change of about 1 to about 50 nucleotides.

“Subgenomic interval” as that term is used herein, refers to a portion of genomic sequence. In an embodiment, a subgenomic interval can be a single nucleotide position, e.g., a variant at the position is associated (positively or negatively) with a tumor phenotype. In an embodiment, a subgenomic interval comprises more than one nucleotide position. Such embodiments include sequences of at least 2, 5, 10, 50, 100, 150, or 250 nucleotide positions in length. Subgenomic intervals can comprise an entire gene, or a portion thereof, e.g., the coding region (or portions thereof), an intron (or portion thereof) or exon (or portion thereof). A subgenomic interval can comprise all or a part of a fragment of a naturally occurring, e.g., genomic DNA, nucleic acid. E.g., a subgenomic interval can correspond to a fragment of genomic DNA which is subjected to a sequencing reaction. In an embodiment, a subgenomic interval is continuous sequence from a genomic source. In an embodiment, a subgenomic interval includes sequences that are not contiguous in the genome, e.g., subgenomic intervals in cDNA can include exon-exon junctions formed as a result of splicing. In an embodiment, the subgenomic interval comprises a tumor nucleic acid molecule. In an embodiment, the subgenomic interval comprises a non-tumor nucleic acid molecule.

In an embodiment, a subgenomic interval comprises or consists of: a single nucleotide position; an intragenic region or an intergenic region; an exon or an intron, or a fragment thereof, typically an exon sequence or a fragment thereof; a coding region or a non-coding region, e.g., a promoter, an enhancer, a 5′ untranslated region (5′ UTR), or a 3′ untranslated region (3′ UTR), or a fragment thereof; a cDNA or a fragment thereof; an SNP; a somatic mutation, a germline mutation or both; an alteration, e.g., a point or a single mutation; a deletion mutation (e.g., an in-frame deletion, an intragenic deletion, a full gene deletion); an insertion mutation (e.g., intragenic insertion); an inversion mutation (e.g., an intra-chromosomal inversion); an inverted duplication mutation; a tandem duplication (e.g., an intrachromosomal tandem duplication); a translocation (e.g., a chromosomal translocation, a non-reciprocal translocation); a rearrangement (e.g., a genomic rearrangement (e.g., a rearrangement of one or more introns, a rearrangement of one or more exons, or a combination and/or a fragment thereof; a rearranged intron can include a 5′- and/or 3′-UTR)); a change in gene copy number; a change in gene expression; a change in RNA levels; or a combination thereof. The “copy number of a gene” refers to the number of DNA sequences in a cell encoding a particular gene product. Generally, for a given gene, a mammal has two copies of each gene. The copy number can be increased, e.g., by gene amplification or duplication, or reduced by deletion.

“Subject interval”, as that term is used herein, refers to a subgenomic interval or an expressed subgenomic interval. In an embodiment, a subgenomic interval and an expressed subgenomic interval correspond, meaning that the expressed subgenomic interval comprises sequence expressed from the corresponding subgenomic interval. In an embodiment, a subgenomic interval and an expressed subgenomic interval are non-corresponding, meaning that the expressed subgenomic interval does not comprise sequence expressed from the non-corresponding subgenomic interval, but rather corresponds to a different subgenomic interval. In an embodiment, a subgenomic interval and an expressed subgenomic interval partially correspond, meaning that the expressed subgenomic interval comprises sequence expressed from the corresponding subgenomic interval and sequence expressed from a different corresponding subgenomic interval.

As used herein, the term “library” refers to a collection of nucleic acid molecules. In one embodiment, the library includes a collection of nucleic acid nucleic acid molecules, e.g., a collection of whole genomic, subgenomic fragments, cDNA, cDNA fragments, RNA, e.g., mRNA, RNA fragments, or a combination thereof. Typically, a nucleic acid molecule is a DNA molecule, e.g., genomic DNA or cDNA. A nucleic acid molecule can be fragmented, e.g., sheared or enzymatically prepared, genomic DNA. Nucleic acid molecules comprise sequence from a subject and can also comprise sequence not derived from the subject, e.g., an adapter sequence, a primer sequence, or other sequences that allow for identification, e.g., “barcode” sequences. In one embodiment, a portion or all of the library nucleic acid molecules comprises an adapter sequence. The adapter sequence can be located at one or both ends. The adapter sequence can be useful, e.g., for a sequencing method (e.g., an NGS method), for amplification, for reverse transcription, or for cloning into a vector. The library can comprise a collection of nucleic acid molecules, e.g., a target nucleic acid molecule (e.g., a tumor nucleic acid molecule, a reference nucleic acid molecule, or a combination thereof). The nucleic acid molecules of the library can be from a single individual. In embodiments, a library can comprise nucleic acid molecules from more than one subject (e.g., 2, 3, 4, 5, 6, 7, 8, 9, 10, 20, 30 or more subjects), e.g., two or more libraries from different subjects can be combined to form a library comprising nucleic acid molecules from more than one subject. In one embodiment, the subject is a human having, or at risk of having, a cancer or tumor.

“Complementary” refers to sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In certain embodiments, the first region comprises a first portion and the second region comprises a second portion, whereby, when the first and second portions are arranged in an antiparallel fashion, at least about 50%, at least about 75%, at least about 90%, or at least about 95% of the nucleotide residues of the first portion are capable of base pairing with nucleotide residues in the second portion. In other embodiments, all nucleotide residues of the first portion are capable of base pairing with nucleotide residues in the second portion.

“Likely to” or “increased likelihood,” as used herein, refers to an increased probability that an item, object, thing or person will occur. Thus, in one example, a subject that is likely to respond to treatment has an increased probability of responding to treatment relative to a reference subject or group of subjects.

“Unlikely to” refers to a decreased probability that an event, item, object, thing or person will occur with respect to a reference. Thus, a subject that is unlikely to respond to treatment has a decreased probability of responding to treatment relative to a reference subject or group of subjects.

“Next-generation sequencing” or “NGS” or “NG sequencing” as used herein, refers to any sequencing method that determines the nucleotide sequence of either individual nucleic acid molecules (e.g., in single molecule sequencing) or clonally expanded proxies for individual nucleic acid molecules in a high throughput fashion (e.g., greater than 103, 104, 105 or more molecules are sequenced simultaneously). In one embodiment, the relative abundance of the nucleic acid species in the library can be estimated by counting the relative number of occurrences of their cognate sequences in the data generated by the sequencing experiment. Next-generation sequencing methods are known in the art, and are described, e.g., in Metzker, M. (2010) Nature Biotechnology Reviews 11:31-46, incorporated herein by reference. Next-generation sequencing can detect a variant present in less than 5% or less than 1% of the nucleic acids in a sample.

“Nucleotide value” as referred herein, represents the identity of the nucleotide(s) occupying or assigned to a nucleotide position. Typical nucleotide values include: missing (e.g., deleted); additional (e.g., an insertion of one or more nucleotides, the identity of which may or may not be included); or present (occupied); A; T; C; or G. Other values can be, e.g., not Y, wherein Y is A, T, G, or C; A or X, wherein X is one or two of T, G, or C; T or X, wherein X is one or two of A, G, or C; G or X, wherein X is one or two of T, A, or C; C or X, wherein X is one or two of T, G, or A; a pyrimidine nucleotide; or a purine nucleotide. A nucleotide value can be a frequency for 1 or more, e.g., 2, 3, or 4, bases (or other value described herein, e.g., missing or additional) at a nucleotide position. E.g., a nucleotide value can comprise a frequency for A, and a frequency for G, at a nucleotide position.

“Or” is used herein to mean, and is used interchangeably with, the term “and/or”, unless context clearly indicates otherwise. The use of the term “and/or” in some places herein does not mean that uses of the term “or” are not interchangeable with the term “and/or” unless the context clearly indicates otherwise.

A “control nucleic acid” or “reference nucleic acid” as used herein, refers to nucleic acid molecules from a control or reference sample. Typically, it is DNA, e.g., genomic DNA, or cDNA derived from RNA, not containing the alteration or variation in the gene or gene product. In certain embodiments, the reference or control nucleic acid sample is a wild-type or a non-mutated sequence. In certain embodiments, the reference nucleic acid sample is purified or isolated (e.g., it is removed from its natural state). In other embodiments, the reference nucleic acid sample is from a blood control, a normal adjacent tissue (NAT), or any other non-cancerous sample from the same or a different subject. In some embodiments, the reference nucleic acid sample comprises normal DNA mixtures. In some embodiments, the normal DNA mixture is a process matched control. In some embodiments, the reference nucleic acid sample has germline variants. In some embodiments, the reference nucleic acid sample does not have somatic alterations, e.g., serves as a negative control.

“Threshold value,” as used herein, is a value that is a function of the number of reads required to be present to assign a nucleotide value to a subject interval (e.g., a subgenomic interval or an expressed subgenomic interval). E.g., it is a function of the number of reads having a specific nucleotide value, e.g., “A,” at a nucleotide position, required to assign that nucleotide value to that nucleotide position in the subgenomic interval. The threshold value can, e.g., be expressed as (or as a function of) a number of reads, e.g., an integer, or as a proportion of reads having the value. By way of example, if the threshold value is X, and X+1 reads having the nucleotide value of “A” are present, then the value of “A” is assigned to the position in the subject interval (e.g., subgenomic interval or expressed subgenomic interval). The threshold value can also be expressed as a function of a mutation or variant expectation, mutation frequency, or of Bayesian prior. In an embodiment, a mutation frequency would require a number or proportion of reads having a nucleotide value, e.g., A or G, at a position, to call that nucleotide value. In embodiments the threshold value can be a function of mutation expectation, e.g., mutation frequency, and tumor type. E.g., a variant at a nucleotide position could have a first threshold value if the patient has a first tumor type and a second threshold value if the patient has a second tumor type.

As used herein, “target nucleic acid molecule” refers to a nucleic acid molecule that one desires to isolate from the nucleic acid library. In one embodiment, the target nucleic acid molecules can be a tumor nucleic acid molecule, a reference nucleic acid molecule, or a control nucleic acid molecule, as described herein.

“Tumor nucleic acid molecule,” or other similar term (e.g., a “tumor or cancer-associated nucleic acid molecule”), as used herein refers to a nucleic acid molecule having sequence from a tumor cell. The terms “tumor nucleic acid molecule” and “tumor nucleic acid” may sometimes be used interchangeably herein. In one embodiment, the tumor nucleic acid molecule includes a subject interval having a sequence (e.g., a nucleotide sequence) that has an alteration (e.g., a mutation) associated with a cancerous phenotype. In other embodiments, the tumor nucleic acid molecule includes a subject interval having a wild-type sequence (e.g., a wild-type nucleotide sequence). For example, a subject interval from a heterozygous or homozygous wild-type allele present in a cancer cell. A tumor nucleic acid molecule can include a reference nucleic acid molecule. Typically, it is DNA, e.g., genomic DNA, or cDNA derived from RNA, from a sample. In certain embodiments, the sample is purified or isolated (e.g., it is removed from its natural state). In some embodiments, the tumor nucleic acid molecule is a cfDNA. In some embodiments, the tumor nucleic acid molecule is a ctDNA. In some embodiments, the tumor nucleic acid molecule is DNA from a CTC.

“Variant,” as used herein, refers to a structure that can be present at a subgenomic interval that can have more than one structure, e.g., an allele at a polymorphic locus.

An “isolated” nucleic acid molecule is one which is separated from other nucleic acid molecules which are present in the natural source of the nucleic acid molecule. In certain embodiments, an “isolated” nucleic acid molecule is free of sequences (such as protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, less than about 4 kB, less than about 3 kB, less than about 2 kB, less than about 1 kB, less than about 0.5 kB or less than about 0.1 kB of nucleotide sequences which naturally flank the nucleic acid molecule in genomic DNA of the cell from which the nucleic acid is derived. Moreover, an “isolated” nucleic acid molecule, such as an RNA molecule or a cDNA molecule, can be substantially free of other cellular material or culture medium, e.g., when produced by recombinant techniques, or substantially free of chemical precursors or other chemicals, e.g., when chemically synthesized.

### III. Individuals to be Treated, Assessed, and Identified

The methods of the present disclosure pertain, in certain embodiments, to individuals having a gastroesophageal cancer and/or samples (such as tumor biopsy samples and/or blood samples) obtained from an individual having a gastroesophageal cancer. In some embodiments, the methods provide improved treatments for the individuals, based on determining a TMB score in a sample obtained from the individual. In some embodiments, the methods provide improved methods of selecting a treatment, identifying one or more treatment options, stratifying the individual for treatment with a therapy, predicting survival of the individual, and/or monitoring, evaluating, or screening the individual, each based in part on determining a TMB score of a sample obtained from the individual.

In some embodiments, the individual has a gastroesophageal cancer. In some embodiments, the individual has been, or is being treated, for cancer. In some embodiments, the individual is in need of being monitored for cancer progression or regression, e.g., after being treated with a cancer therapy. In some embodiments, the individual is in need of being monitored for relapse of cancer. In some embodiments, the individual is at risk of having a cancer. In some embodiments, the individual is suspected of having cancer. In some embodiments, the individual is being tested for cancer. In some embodiments, the individual has a genetic predisposition to a cancer (e.g., having a mutation that increases his or her baseline risk for developing a cancer). In some embodiments, the individual has been exposed to an environment (e.g., radiation or chemical) that increases his or her risk for developing a cancer. In some embodiments, the individual is in need of being monitored for development of a cancer. In some embodiments, the individual is in need of a first line treatment for the cancer.

In certain embodiments, the sample is from an individual having a gastroesophageal cancer. In some embodiments, the cancer is advanced gastroesophageal cancer. In some embodiments, the cancer is metastatic gastroesophageal cancer.

In certain embodiments, the sample is from an individual having a solid tumor or a metastatic form thereof. In certain embodiments, the sample is obtained from a subject having a gastroesophageal cancer, or at risk of having a gastroesophageal cancer. In certain embodiments, the sample is obtained from an individual who has not received a therapy to treat a gastroesophageal cancer as described herein. Premaligancy, as used herein, refers to a tissue that is not yet malignant but is poised to become malignant.

In some embodiments, the individual has not previously received, or is not currently receiving, a treatment for the cancer. In some embodiments, the individual has not previously received a regimen of chemotherapy. In some embodiments, the individual has not received a chemotherapy for the cancer. In some embodiments, the individual is in need of a first line therapy for the cancer.

In some embodiments, the individual is a human. In some embodiments, the individual is a non-human mammal.

### IV. Samples and Processing

The methods of the present disclosure involve, in certain embodiments, determining or assessing a feature (such as a TMB score) of a sample obtained from an individual having a gastroesophageal cancer. In some embodiments, the sample is associated with the gastroesophageal cancer to be treated or assessed. In some embodiments, the sample is from a solid tumor (e.g., a tumor biopsy sample). In some embodiments, the sample is from a liquid sample (e.g., a liquid biopsy sample). In some embodiments, the sample is from a blood sample.

In some embodiments, the sample is associated with gastroesophageal cancer. In some embodiments, the sample is associated with advanced gastroesophageal cancer. In some embodiments, the sample is associated with metastatic gastroesophageal cancer.

In some embodiments, the sample comprises a nucleic acid, e.g., DNA, RNA, or both. In certain embodiments, the sample comprises one or more nucleic acids from a gastroesophageal cancer. In certain embodiments, the sample further comprises one or more non-nucleic acid components from the tumor, e.g., a cell, protein, carbohydrate, or lipid. In certain embodiments, the sample further comprises one or more nucleic acids from a non-tumor cell or tissue.

In some embodiments, the sample comprises one or more nucleic acids, e.g., DNA, RNA, or both, from a premalignant or malignant cell, a cell from a solid tumor, a soft tissue tumor or a metastatic lesion, a histologically normal cell, a circulating tumor cells (CTCs), or a combination thereof. In some embodiments, the sample comprises one or more cells chosen from a premalignant or malignant cell, a cell from a solid tumor, a soft tissue tumor or a metastatic lesion, a histologically normal cell, a circulating tumor cell (CTC), or a combination thereof.

In some embodiments, the sample comprises RNA (e.g., mRNA), DNA, circulating tumor DNA (ctDNA), cell-free DNA (cfDNA), or cell-free RNA (cfRNA) from the gastroesophageal cancer. In some embodiments, the sample comprises cell-free DNA (cfDNA). In some embodiments, cfDNA comprises DNA from healthy tissue, e.g., non-diseased cells, or tumor tissue, e.g., tumor cells. In some embodiments cfDNA from tumor tissue comprises circulating tumor DNA (ctDNA). In some embodiments, the sample further comprises a non-nucleic acid component, e.g., a cell, protein, carbohydrate, or lipid, e.g., from the tumor.

In some embodiments, the sample is from a solid tumor (e.g., a tumor biopsy sample) that comprises tumor-infiltrating immune cells, tumor cells, stromal cells, or any combinations thereof. In some embodiments, the solid tumor sample is a formalin-fixed and paraffin-embedded (FFPE) tumor sample, an archival tumor sample, a fresh tumor sample, a frozen tumor sample, or a core or fine needle tumor biopsy.

In some embodiments, the sample is a liquid sample that comprises blood, plasma, serum, cerebrospinal fluid, sputum, stool, urine, or saliva. In some embodiments, the sample comprises blood, plasma or serum. In certain embodiments, the sample comprises cerebral spinal fluid (CSF). In certain embodiments, the sample comprises pleural effusion. In certain embodiments, the sample comprises ascites. In certain embodiments, the sample comprises urine.

In some embodiments, the sample comprises or is from a blood sample, e.g., is or is from a peripheral whole blood sample. In some embodiments, the peripheral whole blood sample is collected in, e.g., two tubes, e.g., with about 8.5 ml blood per tube. In some embodiments, the peripheral whole blood sample is collected by venipuncture, e.g., according to CLSI H3-A6. In some embodiments, the blood is immediately mixed after collecting, e.g., by gentle inversion, for, e.g., about 8-10 times. In some embodiments, inversion is performed by a complete, e.g., full, 180° turn, e.g., of the wrist. In some embodiments, the blood sample is shipped, e.g., at ambient temperature, e.g., 43-99° F. or 6-37° C. on the same day as collection. In some embodiments, the blood sample is not frozen or refrigerated. In some embodiments, the collected blood sample is kept, e.g., stored, at 43-99° F. or 6-37° C.

In some embodiments of the methods described herein, the method further comprise isolating nucleic acids from a sample described herein. In some embodiments of the methods described herein, the method includes isolating nucleic acids from a sample to provide an isolated nucleic acid sample. In an embodiment, the method includes isolating nucleic acids from a control to provide an isolated control nucleic acid sample. In an embodiment, a method further comprises rejecting a sample with no detectable nucleic acid.

In some embodiments of the methods described herein, the method further comprises acquiring a value for nucleic acid yield in said sample and comparing the acquired value to a reference criterion, e.g., wherein if said acquired value is less than said reference criterion, then amplifying the nucleic acid prior to library construction. In an embodiment, a method further comprises acquiring a value for the size of nucleic acid fragments in said sample and comparing the acquired value to a reference criterion, e.g., a size, e.g., average size, of at least 300, 600, or 900 bps. A parameter described herein can be adjusted or selected in response to this determination.

In some embodiments, the nucleic acids are isolated when they are partially purified or substantially purified. In some embodiments, a nucleic acid is isolated when purified away from other cellular components (e.g., proteins, carbohydrates, or lipids) or other contaminants by standard techniques.

Protocols for DNA isolation from a sample are known in the art, e.g., as provided in Example 1 of U.S. Pat. No. 9,340,830, the content of which is herein incorporated by reference in its entirety. Additional methods to isolate nucleic acids (e.g., DNA) from formaldehyde- or paraformaldehyde-fixed, paraffin-embedded (FFPE) tissues are disclosed, e.g., in Cronin M. et al., (2004) Am J Pathol. 164 (1): 35-42; Masuda N. et al., (1999) Nucleic Acids Res. 27 (22): 4436-4443; Specht K. et al., (2001) Am J Pathol. 158 (2): 419-429, Ambion RecoverAll™ Total Nucleic Acid Isolation Protocol (Ambion, Cat. No. AM1975, September 2008), Maxwell® 16 FFPE Plus LEV DNA Purification Kit Technical Manual (Promega Literature #TM349, February 2011), E.Z.N.A.® FFPE DNA Kit Handbook (OMEGA bio-tek, Norcross, GA, product numbers D3399-00, D3399-01, and D3399-02; June 2009), and QIAamp® DNA FFPE Tissue Handbook (Qiagen, Cat. No. 37625, October 2007). RecoverAll™ Total Nucleic Acid Isolation Kit uses xylene at elevated temperatures to solubilize paraffin-embedded samples and a glass-fiber filter to capture nucleic acids. Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA. Protocols for DNA isolation from blood are disclosed, e.g., in the Maxwell® 16 LEV Blood DNA Kit and Maxwell 16 Buccal Swab LEV DNA Purification Kit Technical Manual (Promega Literature #TM333, Jan. 1, 2011).

Protocols for RNA isolation are disclosed, e.g., in the Maxwell® 16 Total RNA Purification Kit Technical Bulletin (Promega Literature #TB351, August 2009).

The isolated nucleic acids (e.g., genomic DNA) can be fragmented or sheared by practicing routine techniques. For example, genomic DNA can be fragmented by physical shearing methods, enzymatic cleavage methods, chemical cleavage methods, and other methods well known to those skilled in the art. The nucleic acid library can contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library is a portion of the genome, e.g., where the complexity of the genome is reduced by design. In some embodiments, any selected portion of the genome can be used with a method described herein. In certain embodiments, the entire exome or a subset thereof is isolated.

In certain embodiments, the method further includes isolating nucleic acids from the sample to provide a library (e.g., a nucleic acid library as described herein). In certain embodiments, the sample includes whole genomic, subgenomic fragments, or both. The isolated nucleic acids can be used to prepare nucleic acid libraries. Protocols for isolating and preparing libraries from whole genomic or subgenomic fragments are known in the art (e.g., Illumina's genomic DNA sample preparation kit). In certain embodiments, the genomic or subgenomic DNA fragment is isolated from a subject's sample (e.g., a sample described herein).

In still other embodiments, the nucleic acids used to generate the library include RNA or cDNA derived from RNA. In some embodiments, the RNA includes total cellular RNA. In other embodiments, certain abundant RNA sequences (e.g., ribosomal RNAs) have been depleted. In some embodiments, the poly(A)-tailed mRNA fraction in the total RNA preparation has been enriched. In some embodiments, the cDNA is produced by random-primed cDNA synthesis methods. In other embodiments, the cDNA synthesis is initiated at the poly(A) tail of mature mRNAs by priming by oligo (dT)-containing oligonucleotides. Methods for depletion, poly(A) enrichment, and cDNA synthesis are well known to those skilled in the art.

In other embodiments, the nucleic acids are fragmented or sheared by a physical or enzymatic method, and optionally, ligated to synthetic adapters, size-selected (e.g., by preparative gel electrophoresis) and amplified (e.g., by PCR). Alternative methods for DNA shearing are known in the art, e.g., as described in Example 4 in U.S. Pat. No. 9,340,830. For example, alternative DNA shearing methods can be more automatable and/or more efficient (e.g., with degraded FFPE samples). Alternatives to DNA shearing methods can also be used to avoid a ligation step during library preparation.

In other embodiments, the isolated DNA (e.g., the genomic DNA) is fragmented or sheared. In some embodiments, the library includes less than 50% of genomic DNA, such as a subfraction of genomic DNA that is a reduced representation or a defined portion of a genome, e.g., that has been subfractionated by other means. In other embodiments, the library includes all or substantially all genomic DNA.

In other embodiments, the fragmented and adapter-ligated group of nucleic acids is used without explicit size selection or amplification prior to hybrid selection. In some embodiments, the nucleic acid is amplified by a specific or non-specific nucleic acid amplification method that is well known to those skilled in the art. In some embodiments, the nucleic acid is amplified, e.g., by a whole-genome amplification method such as random-primed strand-displacement amplification.

The methods described herein can be performed using a small amount of nucleic acids, e.g., when the amount of source DNA or RNA is limiting (e.g., even after whole-genome amplification). In one embodiment, the nucleic acid comprises less than about 5 μg, 4 μg, 3 μg, 2 μg, 1 μg, 0.8 μg, 0.7 μg, 0.6 μg, 0.5 μg, or 400 ng, 300 ng, 200 ng, 100 ng, 50 ng, 10 ng, 5 ng, 1 ng, or less of nucleic acid sample. For example, one can typically begin with 50-100 ng of genomic DNA. One can start with less, however, if one amplifies the genomic DNA (e.g., using PCR) before the hybridization step, e.g., solution hybridization. Thus it is possible, but not essential, to amplify the genomic DNA before hybridization, e.g., solution hybridization.

In some embodiments, sequencing comprises providing a plurality of nucleic acid molecules obtained from the sample; amplifying nucleic acid molecules from the plurality of nucleic acid molecules; capturing nucleic acid molecules from the amplified nucleic acid molecules; and sequencing, by a sequencer, the captured nucleic acid molecules to obtain a plurality of sequence reads corresponding to one or more genomic loci within a subgenomic interval in the sample. In some embodiments, the plurality of nucleic acid molecules comprises a mixture of tumor nucleic acid molecules and non-tumor nucleic acid molecules.

In some embodiments, amplification of the nucleic acid molecules is performed by a polymerase chain reaction (PCR) technique, a non-PCR amplification technique, or an isothermal amplification technique.

In some embodiments, sequencing further comprises ligating one or more adapters onto one or more nucleic acid molecules from the plurality of nucleic acid molecules. In some embodiments, the adapters comprise one or more of amplification primer sequences, flow cell adapter hybridization sequences, unique molecular identifier sequences, substrate adapter sequences, or sample index sequences.

In some embodiments, nucleic acid molecules from a library are isolated, e.g., using solution hybridization, thereby providing a library catch. The library catch, or a subgroup thereof, can be sequenced. Accordingly, the methods described herein can further include analyzing the library catch. In some embodiments, the library catch is analyzed by a sequencing method, e.g., a next-generation sequencing method as described herein. In some embodiments, the method includes isolating a library catch by solution hybridization, and subjecting the library catch to nucleic acid sequencing. In certain embodiments, the library catch is re-sequenced.

In some embodiments, the captured nucleic acid molecules are captured from the amplified nucleic acid molecules by hybridization to one or more bait molecules. In some embodiments, the one or more bait molecules comprise one or more nucleic acid molecules, each comprising a region that is complementary to a region of a captured nucleic acid molecule. In some embodiments, the one or more bait molecules each comprise a capture moiety. In some embodiments, the capture moiety is biotin.

Any method of sequencing known in the art can be used. Sequencing of nucleic acids, e.g., isolated by solution hybridization, are typically carried out using next-generation sequencing (NGS). Sequencing methods suitable for use herein are described in the art, e.g., as described in U.S. Pat. No. 9,340,830. In some embodiments, sequencing is performed using a massively parallel sequencing (MPS) technique, whole genome sequencing (WGS), whole exome sequencing (WES), targeted sequencing, direct sequencing, next-generation sequencing (NGS), or a Sanger sequencing technique.

In some embodiments, sequencing comprises detecting alterations present in the genome, whole exome or transcriptome of an individual. In some embodiments, sequencing comprises DNA and/or RNA sequencing, e.g., targeted DNA and/or RNA sequencing. In some embodiments, the sequencing comprises detection of a change (e.g., an increase or decrease) in the level of a gene or gene product, e.g., a change in expression of a gene or gene product described herein.

Sequencing can, optionally, include a step of enriching a sample for a target RNA. In other embodiments, sequencing includes a step of depleting the sample of certain high abundance RNAs, e.g., ribosomal or globin RNAs. The RNA sequencing methods can be used, alone or in combination with the DNA sequencing methods described herein. In one embodiment, sequencing includes a DNA sequencing step and an RNA sequencing step. The methods can be performed in any order. For example, the method can include confirming by RNA sequencing the expression of an alteration described herein, e.g., confirming expression of a mutation or a fusion detected by the DNA sequencing methods of the invention. In other embodiments, sequencing includes performing an RNA sequencing step, followed by a DNA sequencing step.

In some embodiments, the sample is associated with a gastroesophageal cancer that has not been previously treated with an anti-cancer therapy. In some embodiments, the sample is associated with a gastroesophageal cancer that has not been previously treated with a chemotherapy.

In some embodiments, the sample is a mammalian sample. In some embodiments, the sample is a human sample. In some embodiments, the sample is a non-human mammalian sample.

### V. Methods for Determining Tumor Mutational Burden

Tumor mutational burden (TMB) is, broadly, the number of somatic mutations per megabase of genomic region. It has been discovered that a TMB score (which can be expressed, for example, as mutations per megabase, i.e., mutations/Mb) can inform treatment decisions, including, in some embodiments, whether to administer an immune checkpoint inhibitor therapy without a chemotherapy regimen to a patient if a TMB score is at or higher than a threshold TMB score or administer an immune checkpoint inhibitor therapy with a chemotherapy regimen if a TMB score is below a threshold TMB score. The TMB score can be determined using a variety of techniques including next-generation sequencing (NGS) techniques.

In some embodiments, the methods of the disclosure comprise determining a tumor mutational burden (TMB) score in a sample obtained from an individual. In some embodiments, the sample from the individual comprises fluid, cells, or tissue. In some embodiments, the sample from the individual comprises a tumor biopsy or a circulating tumor cell. In some embodiments, the sample from the individual comprises nucleic acids. In some embodiments, the sample from the individual comprises mRNA, DNA, circulating tumor DNA, cell-free DNA, or cell-free RNA.

In some embodiments, the TMB score is determined by sequencing. In some embodiments, the TMB score is determined by sequencing using a high-throughput sequencing technique, such as next-generation sequencing (NGS), an NGS-based method, or an NGS-derived method. In some embodiments, the NGS method is selected from whole genome sequencing (WGS), whole exome sequencing (WES) or a comprehensive genomic profiling (CGP). In some embodiments, the sequencing comprises sequencing a panel of cancer genes. In some embodiments, the TMB score reflects the number of nonsynonymous mutations, such as missense mutations or nonsense mutations, in a sequence. In some embodiments, the TMB score is determined by normalizing a matched tumor biopsy sample sequence with germline sequences to exclude inherited germline mutations.

The “threshold TMB score” described herein refers to a predetermined TMB score which a measured TMB score (i.e., the TMB score determined in a sample from an individual having a gastroesophageal cancer) is compared with. Comparison of the determined TMB score with the threshold TMB score is used, in certain embodiments, to inform treatment decisions or identify treatment options for an individual. In some embodiments, the threshold TMB score is at or higher than 8 mutations/Mb, such as at or higher than any of about 9 mutations/Mb, about 10 mutations/Mb, about 11 mutations/Mb, about 12 mutations/Mb, about 13 mutations/Mb, about 14 mutations/Mb, about 15 mutations/Mb, about 16 mutations/Mb, about 17 mutations/Mb, about 18 mutations/Mb, about 19 mutations/Mb, about 20 mutations/Mb, about 21 mutations/Mb, about 22 mutations/Mb, about 23 mutations/Mb, about 24 mutations/Mb, about 25 mutations/Mb, about 26 mutations/Mb, about 27 mutations/Mb, about 28 mutations/Mb, about 29 mutations/Mb, about 30 mutations/Mb, about 31 mutations/Mb, about 32 mutations/Mb, about 33 mutations/Mb, about 34 mutations/Mb, about 35 mutations/Mb, about 36 mutations/Mb, about 37 mutations/Mb, about 38 mutations/Mb, about 39 mutations/Mb, about 40 mutations/Mb, about 41 mutations/Mb, about 42 mutations/Mb, about 43 mutations/Mb, about 44 mutations/Mb, about 45 mutations/Mb, about 46 mutations/Mb, about 47 mutations/Mb, about 48 mutations/Mb, about 49 mutations/Mb, or about 50 mutations/Mb. In some embodiments, the threshold TMB score is about 8 mutations/Mb, about 9 mutations/Mb, about 10 mutations/Mb, about 11 mutations/Mb, about 12 mutations/Mb, about 13 mutations/Mb, about 14 mutations/Mb, about 15 mutations/Mb, about 16 mutations/Mb, about 17 mutations/Mb, about 18 mutations/Mb, about 19 mutations/Mb, about 20 mutations/Mb, about 21 mutations/Mb, about 22 mutations/Mb, about 23 mutations/Mb, about 24 mutations/Mb, about 25 mutations/Mb, about 26 mutations/Mb, about 27 mutations/Mb, about 28 mutations/Mb, about 29 mutations/Mb, about 30 mutations/Mb, about 31 mutations/Mb, about 32 mutations/Mb, about 33 mutations/Mb, about 34 mutations/Mb, about 35 mutations/Mb, about 36 mutations/Mb, about 37 mutations/Mb, about 38 mutations/Mb, about 39 mutations/Mb, about 40 mutations/Mb, about 41 mutations/Mb, about 42 mutations/Mb, about 43 mutations/Mb, about 44 mutations/Mb, about 45 mutations/Mb, about 46 mutations/Mb, about 47 mutations/Mb, about 48 mutations/Mb, about 49 mutations/Mb, or about 50 mutations/Mb. In some embodiments, the threshold TMB score is about 10 mutations/Mb. In some embodiments, the threshold TMB score is 10 mutations/Mb. In some embodiments, the threshold TMB score is a “high tumor mutational burden score,” e.g., a TMB score of at or higher than about 10 mutations/Mb, such as any of at or higher than about 15 mutations/Mb, about 20 mutations/Mb, about 25 mutations/Mb, about 30 mutations/Mb, about 35 mutations/Mb, about 40 mutations/Mb, about 45 mutations/Mb, about 50 mutations/Mb, or more.

In some embodiments, tumor mutational burden is measured according to the methods provided in U.S. patent application No. 20180363066, which is hereby incorporated by reference in its entirety. In some embodiments, tumor mutational burden is measured according to the methods described in Montesion, M., et al., Cancer Discovery (2021) 11 (2): 282-92. In some embodiments, tumor mutational burden is measured according to the methods described in Chalmers et al., “Analysis of 100,00 human cancer genomes reveals the landscape of tumor mutational burden,” Genome Med. 2017; 9 (1): 34). In some embodiments, the tumor mutational burden is measured according to the methods described in Huang, R., et al., “Durable responders in advanced NSCLC with elevated TMB and treated with 1 L immune checkpoint inhibitor: a real-world outcomes analysis,” J Immunother Cancer (2023) 11 (1): e005801. In some embodiments, the tumor mutational burden is measured according to the methods described in Quintanilha, J., et al., “Comparative Effectiveness of Immune Checkpoint Inhibitors vs Chemotherapy in Patients With Metastatic Colorectal Cancer With Measures of Microsatellite Instability, Mismatch Repair, or Tumor Mutational Burden,” JAMA Netw Open. (2023) 6 (1): e2252244.

In some embodiments, tumor mutational burden is assessed based on the number of non-driver somatic coding mutations/megabase (mut/Mb) of genome sequenced.

In some embodiments, tumor mutational burden is measured in the sample using next-generation sequencing. In some embodiments, tumor mutational burden is measured in the sample by whole exome sequencing. In some embodiments, tumor mutational burden is measured in the sample using whole genome sequencing. In some embodiments, tumor mutational burden is measured in the sample by gene-targeted sequencing. In some embodiments, tumor mutational burden is measured on between about 0.8 Mb and about 1.3 Mb of sequenced DNA. In some embodiments, tumor mutational burden is measured on any of about 0.8 Mb, about 0.81 Mb, about 0.82 Mb, about 0.83 Mb, about 0.84 Mb, about 0.85 Mb, about 0.86 Mb, about 0.87 Mb, about 0.88 Mb, about 0.89 Mb, about 0.9 Mb, about 0.91 Mb, about 0.92 Mb, about 0.93 Mb, about 0.94 Mb, about 0.95 Mb, about 0.96 Mb, about 0.97 Mb, about 0.98 Mb, about 0.99 Mb, about 1 Mb, about 1.01 Mb, about 1.02 Mb, about 1.03 Mb, about 1.04 Mb, about 1.05 Mb, about 1.06 Mb, about 1.07 Mb, about 1.08 Mb, about 1.09 Mb, about 1.1 Mb, about 1.2 Mb, or about 1.3 Mb of sequenced DNA. In some embodiments, tumor mutational burden is measured on about 0.8 Mb of sequenced DNA. In some embodiments, tumor mutational burden is measured on between about 0.83 Mb and about 1.14 Mb of sequenced DNA. In some embodiments, tumor mutational burden is measured on up to about 1.24 Mb of sequenced DNA. In some embodiments, tumor mutational burden is measured on up to about 1.1 Mb of sequenced DNA. In some embodiments, tumor mutational burden is measured on about 0.79 Mb of sequenced DNA.

In some embodiments, the TMB score is less than about 10 mutations/Mb. In some embodiments, the TMB score is more than about 10 mutations/Mb. In some embodiments, the TMB score is at or higher than 10 mutations/Mb. In some embodiments, the TMB score is a high tumor mutational burden score, e.g., of at or higher than about 10 mut/Mb. In some embodiments, the TMB score is at or higher than about 10 mut/Mb. In some embodiments, the TMB score is at or higher than about 20 mut/Mb. In some embodiments, the TMB score is between about 10 mut/Mb and about 15 mut/Mb, between about 15 mut/Mb and about 20 mut/Mb, between about 20 mut/Mb and about 25 mut/Mb, between about 25 mut/Mb and about 30 mut/Mb, between about 30 mut/Mb and about 35 mut/Mb, between about 35 mut/Mb and about 40 mut/Mb, between about 40 mut/Mb and about 45 mut/Mb, between about 45 mut/Mb and about 50 mut/Mb, between about 50 mut/Mb and about 55 mut/Mb, between about 55 mut/Mb and about 60 mut/Mb, between about 60 mut/Mb and about 65 mut/Mb, between about 65 mut/Mb and about 70 mut/Mb, between about 70 mut/Mb and about 75 mut/Mb, between about 75 mut/Mb and about 80 mut/Mb, between about 80 mut/Mb and about 85 mut/Mb, between about 85 mut/Mb and about 90 mut/Mb, between about 90 mut/Mb and about 95 mut/Mb, or between about 95 mut/Mb and about 100 mut/Mb. In some embodiments, the TMB score is between about 100 mut/Mb and about 110 mut/Mb, between about 110 mut/Mb and about 120 mut/Mb, between about 120 mut/Mb and about 130 mut/Mb, between about 130 mut/Mb and about 140 mut/Mb, between about 140 mut/Mb and about 150 mut/Mb, between about 150 mut/Mb and about 160 mut/Mb, between about 160 mut/Mb and about 170 mut/Mb, between about 170 mut/Mb and about 180 mut/Mb, between about 180 mut/Mb and about 190 mut/Mb, between about 190 mut/Mb and about 200 mut/Mb, between about 210 mut/Mb and about 220 mut/Mb, between about 220 mut/Mb and about 230 mut/Mb, between about 230 mut/Mb and about 240 mut/Mb, between about 240 mut/Mb and about 250 mut/Mb, between about 250 mut/Mb and about 260 mut/Mb, between about 260 mut/Mb and about 270 mut/Mb, between about 270 mut/Mb and about 280 mut/Mb, between about 280 mut/Mb and about 290 mut/Mb, between about 290 mut/Mb and about 300 mut/Mb, between about 300 mut/Mb and about 310 mut/Mb, between about 310 mut/Mb and about 320 mut/Mb, between about 320 mut/Mb and about 330 mut/Mb, between about 330 mut/Mb and about 340 mut/Mb, between about 340 mut/Mb and about 350 mut/Mb, between about 350 mut/Mb and about 360 mut/Mb, between about 360 mut/Mb and about 370 mut/Mb, between about 370 mut/Mb and about 380 mut/Mb, between about 380 mut/Mb and about 390 mut/Mb, between about 390 mut/Mb and about 400 mut/Mb, or more than 400 mut/Mb. In some embodiments, the TMB score is at or higher than about 100 mut/Mb, at or higher than about 110 mut/Mb, at or higher than about 120 mut/Mb, at or higher than about 130 mut/Mb, at or higher than about 140 mut/Mb, at or higher than about 150 mut/Mb, or more. In some embodiments, the TMB score is determined based on between about 0.8 Mb to about 1.1 Mb.

In some embodiments, measuring tumor mutational burden comprises assessing mutations in a sample derived from a cancer in an individual. In some embodiments, measuring tumor mutational burden comprises assessing mutations in a sample derived from a cancer in an individual and in a matched normal sample, e.g., a sample from the individual derived from a tissue or other source that is free of the cancer. In some embodiments, TMB is detected directly from one or more nucleic acids from the sample. In some embodiments, TMB is detected from an amplicon, sequence read, or other nucleic acid otherwise derived from one or more nucleic acids from the sample.

In some embodiments, tumor mutational burden is obtained from a plurality of sequence reads, e.g., a plurality of sequence reads obtained by sequencing nucleic acids corresponding to at least a portion of a genome (such as from an enriched or unenriched sample), e.g., according to any sequencing method known in the art or described herein. In some embodiments, tumor mutational burden is determined based on the number of non-driver somatic coding mutations per megabase of genome sequenced.

In some embodiments, the TMB score is a tissue TMB score (tTMB). As used herein, the terms “solid tumor TMB score,” “tissue TMB score,” and “tTMB score,” are used interchangeably and refer to a numerical value that reflects the number of somatic mutations detected in a tumor biopsy sample (e.g., a solid tumor biopsy sample) obtained from an individual (e.g., an individual at risk of or having a gastroesophageal cancer). The tTMB score can be measured, for example, on a whole genome or exome basis, or on the basis of a subset of the genome or exome (e.g., a predetermined set of genes). In some embodiments, the tTMB score can be measured based on intergenic sequences. In some embodiments, the tTMB score measured on the basis of a subset of genome or exome can be extrapolated to determine a whole genome or exome tTMB score. In certain embodiments, the predetermined set of genes does not comprise the entire genome or the entire exome. In other embodiments, the set of subgenomic intervals does not comprise the entire genome or the entire exome. In some embodiments, the predetermined set of genes comprise a plurality of genes, which in mutant form, are associated with an effect on cell division, growth or survival, or are associated with cancer. In some embodiments, the predetermined set of genes comprise at least about 50 or more, about 100 or more, about 150 or more, about 200 or more, about 250 or more, about 300 or more, about 350 or more, about 400 or more, about 450 or more, or about 500 or more genes. In some embodiments, the pre-determined set of genes covers about 1 Mb (e.g., about 1.1 Mb, e.g., about 1.125 Mb). In some embodiments, the number of somatic mutations is the number of single nucleotide variants (SNVs) counted or a sum of the number of SNVs and the number of indel mutations counted. In some embodiments, the tTMB score refers to the number of accumulated somatic mutations in a tumor.

In some embodiments, the TMB score is a blood TMB score (bTMB). As used herein, the terms “blood tumor mutational burden score,” “blood tumor mutation burden score,” and “bTMB score,” each of which may be used interchangeably, refer to a numerical value that reflects the number of somatic mutations detected in cell-free DNA (cfDNA) derived from a blood sample (e.g., a whole blood sample, a plasma sample, a serum sample, or a combination thereof) obtained from an individual (e.g., an individual at risk of or having a gastroesophageal cancer). The bTMB score can be measured, for example, on a whole genome or exome basis, or on the basis of a subset of the genome or exome (e.g., a predetermined set of genes). In certain embodiments, a bTMB score can be measured based on intergenic sequences. In some embodiments, the bTMB score measured on the basis of a subset of genome or exome can be extrapolated to determine a whole genome or exome bTMB score. In certain embodiments, the predetermined set of genes does not comprise the entire genome or the entire exome. In other embodiments, the set of subgenomic intervals does not comprise the entire genome or the entire exome. In some embodiments, the predetermined set of genes comprise a plurality of genes, which in mutant form, are associated with an effect on cell division, growth or survival, or are associated with cancer. In some embodiments, the predetermined set of genes comprise at least about 50 or more, about 100 or more, about 150 or more, about 200 or more, about 250 or more, about 300 or more, about 350 or more, about 400 or more, about 450 or more, or about 500 or more genes. In some embodiments, the pre-determined set of genes covers about 1 Mb (e.g., about 1.1 Mb, e.g., about 1.125 Mb). In some embodiments, the bTMB score is determined from measuring the number of somatic mutations in circulating tumor DNA (ctDNA) in a sample. In some embodiments, the number of somatic mutations is the number of single nucleotide variants (SNVs) counted or a sum of the number of SNVs and the number of indel mutations counted. In some embodiments, the bTMB score refers to the number of accumulated somatic mutations in a tumor.

In some embodiments, bTMB or tTMB is measured using any suitable method known in the art. For example, tumor mutational burden may be measured using whole-exome sequencing (WES), next-generation sequencing, whole genome sequencing, gene-targeted sequencing, or sequencing of a panel of genes, e.g., panels including cancer-related genes. See, e.g., Melendez et al., Transl Lung Cancer Res (2018) 7 (6): 661-667. In some embodiments, tumor mutational burden is measured using gene-targeted sequencing, e.g., using a nucleic acid hybridization-capture method, e.g., coupled with sequencing. See, e.g., Fancello et al., J Immunother Cancer (2019) 7:183.

### VI. Immune Checkpoint Inhibitors

The methods described herein pertain, in certain embodiments, to means of predicting the efficacy of an immune checkpoint inhibitor (ICPI) therapy without a chemotherapy regimen and/or administering an ICPI to an individual having a gastroesophageal cancer. In some embodiments, the efficacy of the ICPI therapy is predicted as a first line treatment. In some embodiments, the ICPI therapy is administered as a first line therapy for a gastroesophageal cancer. In some embodiments, the ICPI therapy is the only treatment administered or indicated. In some embodiments, the ICPI therapy consists of a single active agent, such as a single immune checkpoint inhibitor. In some embodiments, the ICPI therapy is administered or indicated to be administered with another treatment, such as a non-ICPI therapy. In some embodiments, the ICPI therapy comprises a combination ICPI therapy comprising two or more ICPIs, that is, two or more different active agents which target immune checkpoints. In some embodiments, the two or more different active agents each target a different immune checkpoint protein.

As is known in the art, a checkpoint inhibitor targets at least one immune checkpoint protein to alter the regulation of an immune response. Immune checkpoint proteins include, e.g., CTLA4, PD-L1, PD-1, PD-L2, VISTA, B7-H2, B7-H3, B7-H4, B7-H6, 2B4, ICOS, HVEM, CEACAM, LAIR1, CD80, CD86, CD276, VTCN1, MHC class I, MHC class II, GALS, adenosine, TGFR, CSF1R, MICA/B, arginase, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some embodiments, molecules involved in regulating immune checkpoints include, but are not limited to: PD-1 (CD279), PD-L1 (B7-H1, CD274), PD-L2 (B7-CD, CD273), CTLA-4 (CD152), HVEM, BTLA (CD272), a killer-cell immunoglobulin-like receptor (KIR), LAG-3 (CD223), TIM-3 (HAVCR2), CEACAM, CEACAM-1, CEACAM-3, CEACAM-5, GAL9, VISTA (PD-1H), TIGIT, LAIR1, CD160, 2B4, TGFRbeta, A2AR, GITR (CD357), CD80 (B7-1), CD86 (B7-2), CD276 (B7-H3), VTCNI (B7-H4), MHC class I, MHC class II, GALS, adenosine, TGFR, B7-H1, OX40 (CD134), CD94 (KLRD1), CD137 (4-1BB), CD137L (4-1BBL), CD40, IDO, CSF1R, CD40L, CD47, CD70 (CD27L), CD226, HHLA2, ICOS (CD278), ICOSL (CD275), LIGHT (TNFSF14, CD258), NKG2a, NKG2d, OX40L (CD134L), PVR (NECL5, CD155), SIRPa, MICA/B, and/or arginase. In some embodiments, an immune checkpoint inhibitor (i.e., a checkpoint inhibitor) decreases the activity of a checkpoint protein that negatively regulates immune cell function, e.g., in order to enhance T cell activation and/or an anti-cancer immune response. In other embodiments, a checkpoint inhibitor increases the activity of a checkpoint protein that positively regulates immune cell function, e.g., in order to enhance T cell activation and/or an anti-cancer immune response. In some embodiments, the checkpoint inhibitor is an antibody. Examples of checkpoint inhibitors include, without limitation, a PD-1 axis binding antagonist, a PD-L1 axis binding antagonist (e.g., an anti-PD-L1 antibody, e.g., atezolizumab (MPDL3280A)), an antagonist directed against a co-inhibitory molecule (e.g., a CTLA4 antagonist (e.g., an anti-CTLA4 antibody), a TIM-3 antagonist (e.g., an anti-TIM-3 antibody), or a LAG-3 antagonist (e.g., an anti-LAG-3 antibody)), or any combination thereof. In some embodiments, the immune checkpoint inhibitors comprise drugs such as small molecules, recombinant forms of ligand or receptors, or antibodies, such as human antibodies (see, e.g., International Patent Publication WO2015016718; Pardoll, Nat Rev Cancer, 12 (4): 252-64, 2012; both incorporated herein by reference). In some embodiments, known inhibitors of immune checkpoint proteins or analogs thereof may be used, in particular chimerized, humanized or human forms of antibodies may be used.

In some embodiments according to any of the embodiments described herein, the immune checkpoint inhibitor comprises a PD-1 antagonist/inhibitor or a PD-L1 antagonist/inhibitor.

In some embodiments, the checkpoint inhibitor is a PD-L1 axis binding antagonist, e.g., a PD-1 binding antagonist, a PD-L1 binding antagonist, or a PD-L2 binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1 LG1,” “CD274,” “B7-H,” and “PDL1.” An exemplary human PD-L1 is shown in UniProtKB/Swiss-Prot Accession No.Q9NZQ7.1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

In some instances, the PD-1 binding antagonist/inhibitor is a molecule that inhibits the binding of PD-1 to its ligand binding partners. In a specific embodiment, the PD-1 ligand binding partners are PD-L1 and/or PD-L2. In another instance, a PD-L1 binding antagonist/inhibitor is a molecule that inhibits the binding of PD-L1 to its binding ligands. In a specific embodiment, PD-L1 binding partners are PD-1 and/or B7-1. In another instance, the PD-L2 binding antagonist is a molecule that inhibits the binding of PD-L2 to its ligand binding partners. In a specific embodiment, the PD-L2 binding ligand partner is PD-1. The antagonist may be an antibody, an antigen binding fragment thereof, an immunoadhesin, a fusion protein, or an oligopeptide. In some embodiments, the PD-1 binding antagonist is a small molecule, a nucleic acid, a polypeptide (e.g., antibody), a carbohydrate, a lipid, a metal, or a toxin.

In some instances, the PD-1 binding antagonist is an anti-PD-1 antibody (e.g., a human antibody, a humanized antibody, or a chimeric antibody), for example, as described below. In some instances, the anti-PD-1 antibody is MDX-1 106 (nivolumab), MK-3475 (pembrolizumab, Keytruda®), cemiplimab, dostarlimab, MEDI-0680 (AMP-514), PDR001, REGN2810, MGA-012, JNJ-63723283, BI 754091, or BGB-108. In other instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some instances, the PD-1 binding antagonist is AMP-224. Other examples of anti-PD-1 antibodies include, but are not limited to, MEDI-0680 (AMP-514; AstraZeneca), PDR001 (CAS Registry No. 1859072-53-9; Novartis), REGN2810 (LIBTAYO® or cemiplimab-rwlc; Regeneron), BGB-108 (BeiGene), BGB-A317 (BeiGene), BI 754091, JS-001 (Shanghai Junshi), STI-A1110 (Sorrento), INCSHR-1210 (Incyte), PF-06801591 (Pfizer), TSR-042 (also known as ANB011; Tesaro/AnaptysBio), AM0001 (ARMO Biosciences), ENUM 244C8 (Enumeral Biomedical Holdings), or ENUM 388D4 (Enumeral Biomedical Holdings). In some embodiments, the PD-1 axis binding antagonist comprises tislelizumab (BGB-A317), BGB-108, STI-A1110, AM0001, BI 754091, sintilimab (IBI308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), pembrolizumab (MK-3475, SCH 900475, Keytruda®), PF-06801591, cemiplimab (REGN-2810, REGEN2810), dostarlimab (TSR-042, ANB011), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, or CCX-4503, or derivatives thereof.

In some embodiments, the PD-L1 binding antagonist is a small molecule that inhibits PD-1. In some embodiments, the PD-L1 binding antagonist is a small molecule that inhibits PD-L1. In some embodiments, the PD-L1 binding antagonist is a small molecule that inhibits PD-L1 and VISTA or PD-L1 and TIM3. In some embodiments, the PD-L1 binding antagonist is CA-170 (also known as AUPM-170). In some embodiments, the PD-L1 binding antagonist is an anti-PD-L1 antibody. In some embodiments, the anti-PD-L1 antibody can bind to a human PD-L1, for example a human PD-L1 as shown in UniProtKB/Swiss-Prot Accession No.Q9NZQ7.1, or a variant thereof. In some embodiments, the PD-L1 binding antagonist is a small molecule, a nucleic acid, a polypeptide (e.g., antibody), a carbohydrate, a lipid, a metal, or a toxin.

In some instances, the PD-L1 binding antagonist is an anti-PD-L1 antibody, for example, as described below. In some instances, the anti-PD-L1 antibody is capable of inhibiting the binding between PD-L1 and PD-1, and/or between PD-L1 and B7-1. In some instances, the anti-PD-L1 antibody is a monoclonal antibody. In some instances, the anti-PD-L1 antibody is an antibody fragment selected from a Fab, Fab′-SH, Fv, scFv, or (Fab′) 2 fragment. In some instances, the anti-PD-L1 antibody is a humanized antibody. In some instances, the anti-PD-L1 antibody is a human antibody. In some instances, the anti-PD-L1 antibody is selected from YW243.55.S70, MPDL3280A (atezolizumab), MDX-1 105, MEDI4736 (durvalumab), or MSB0010718C (avelumab). In some embodiments, the PD-L1 axis binding antagonist comprises atezolizumab, avelumab, durvalumab (imfinzi), BGB-A333, SHR-1316 (HTI-1088), CK-301, BMS-936559, envafolimab (KN035, ASC22), CS1001, MDX-1105 (BMS-936559), LY3300054, STI-A1014, FAZ053, CX-072, INCB086550, GNS-1480, CA-170, CK-301, M-7824, HTI-1088 (HTI-131, SHR-1316), MSB-2311, AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IMM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, or FS-118, or a derivative thereof.

In some embodiments, the checkpoint inhibitor is an antagonist/inhibitor of CTLA4. In some embodiments, the checkpoint inhibitor is a small molecule antagonist of CTLA4. In some embodiments, the checkpoint inhibitor is an anti-CTLA4 antibody. CTLA4 is part of the CD28-B7 immunoglobulin superfamily of immune checkpoint molecules that acts to negatively regulate T cell activation, particularly CD28-dependent T cell responses. CTLA4 competes for binding to common ligands with CD28, such as CD80 (B7-1) and CD86 (B7-2), and binds to these ligands with higher affinity than CD28. Blocking CTLA4 activity (e.g., using an anti-CTLA4 antibody) is thought to enhance CD28-mediated costimulation (leading to increased T cell activation/priming), affect T cell development, and/or deplete Tregs (such as intratumoral Tregs). In some embodiments, the CTLA4 antagonist is a small molecule, a nucleic acid, a polypeptide (e.g., antibody), a carbohydrate, a lipid, a metal, or a toxin. In some embodiments, the CTLA-4 inhibitor comprises ipilimumab (IBI310, BMS-734016, MDX010, MDX-CTLA4, MEDI4736), tremelimumab (CP-675, CP-675,206), APL-509, AGEN1884, CS1002, AGEN1181, Abatacept (Orencia, BMS-188667, RG2077), BCD-145, ONC-392, ADU-1604, REGN4659, ADG116, KN044, KN046, or a derivative thereof.

In some embodiments, the anti-PD-1 antibody or antibody fragment is MDX-1106 (nivolumab), MK-3475 (pembrolizumab, Keytruda®), cemiplimab, dostarlimab, MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, or ENUM 388D4. In some embodiments, the PD-1 binding antagonist is an anti-PD-1 immunoadhesin. In some embodiments, the anti-PD-1 immunoadhesin is AMP-224. In some embodiments, the anti-PD-L1 antibody or antibody fragment is YW243.55.S70, MPDL3280A (atezolizumab), MDX-1105, MEDI4736 (durvalumab), MSB0010718C (avelumab), LY3300054, STI-A1014, KN035, FAZ053, or CX-072.

In some embodiments, the immune checkpoint inhibitor comprises a LAG-3 inhibitor (e.g., an antibody, an antibody conjugate, or an antigen-binding fragment thereof). In some embodiments, the LAG-3 inhibitor comprises a small molecule, a nucleic acid, a polypeptide (e.g., an antibody), a carbohydrate, a lipid, a metal, or a toxin. In some embodiments, the LAG-3 inhibitor comprises a small molecule. In some embodiments, the LAG-3 inhibitor comprises a LAG-3 binding agent. In some embodiments, the LAG-3 inhibitor comprises an antibody, an antibody conjugate, or an antigen-binding fragment thereof. In some embodiments, the LAG-3 inhibitor comprises eftilagimod alpha (IMP321, IMP-321, EDDP-202, EOC-202), relatlimab (BMS-986016), GSK2831781 (IMP-731), LAG525 (IMP701), TSR-033, EVIP321 (soluble LAG-3 protein), BI 754111, IMP761, REGN3767, MK-4280, MGD-013, XmAb22841, INCAGN-2385, ENUM-006, AVA-017, AM-0003, iOnctura anti-LAG-3 antibody, Arcus Biosciences LAG-3 antibody, Sym022, a derivative thereof, or an antibody that competes with any of the preceding.

In some embodiments, the immune checkpoint inhibitor is monovalent and/or monospecific. In some embodiments, the immune checkpoint inhibitor is multivalent and/or multispecific.

In some embodiments, the immune checkpoint inhibitor may be administered in combination with an immunoregulatory molecule or a cytokine. An immunoregulatory profile is required to trigger an efficient immune response and balance the immunity in a subject. Examples of suitable immunoregulatory cytokines include, but are not limited to, interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFα and TNFβ), erythropoietin (EPO), FLT-3 ligand, glp10, TCA-3, MCP-1, MIF, MIP-1α, MIP-1β, Rantes, macrophage colony stimulating factor (M-CSF), granulocyte colony stimulating factor (G-CSF), or granulocyte-macrophage colony stimulating factor (GM-CSF), as well as functional fragments thereof. In some embodiments, any immunomodulatory chemokine that binds to a chemokine receptor, i.e., a CXC, CC, C, or CX3C chemokine receptor, can be used in the context of the present disclosure. Examples of chemokines include, but are not limited to, MIP-3α (Lax), MIP-3β, Hcc-1, MPIF-1, MPIF-2, MCP-2, MCP-3, MCP-4, MCP-5, Eotaxin, Tarc, Elc, I309, IL-8, GCP-2 Groα, Gro-β, Nap-2, Ena-78, Ip-10, MIG, I-Tac, SDF-1, or BCA-1 (Blc), as well as functional fragments thereof. In some embodiments, the immunoregulatory molecule is included with any of the treatments provided herein.

In some embodiments, the immune checkpoint inhibitor is a first line immune checkpoint inhibitor (e.g., it is a first line therapy for the gastroesophageal cancer). In some embodiments, an immune checkpoint inhibitor is administered in combination with one or more additional anti-cancer therapies or treatments.

### VII. Chemotherapies

The methods described herein pertain, in certain embodiments, to means of predicting the efficacy of an immune checkpoint inhibitor therapy with a chemotherapy regimen and/or administering an immune checkpoint inhibitor therapy with a chemotherapy regimen to an individual having a gastroesophageal cancer. For example, in some embodiments, when a TMB score of a tumor is determined to be below a threshold level, such as below 10 mut/megabase or below 20 mut/megabase, then the tumor is not indicated as a suitable candidate for an ICPI therapy without a chemotherapy regimen and is instead indicated for therapy with an ICPI therapy with a chemotherapy regimen.

In some embodiments, the methods provided herein comprise administering to an individual a chemotherapy. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenishers such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids, such as retinoic acid; capecitabine; carboplatin, procarbazine, plicomycin, gemcitabine, navelbine, farnesyl-protein transferase inhibitors, transplatinum, fluorouracil, a taxane, and pharmaceutically acceptable salts, acids, or derivatives of any of the above.

Some non-limiting examples of chemotherapeutic drugs of the present disclosure are carboplatin (Paraplatin), cisplatin (Platinol, Platinol-AQ), cyclophosphamide (Cytoxan, Neosar), docetaxel (Taxotere), doxorubicin (Adriamycin), erlotinib (Tarceva), etoposide (VePesid), fluorouracil (5-FU), gemcitabine (Gemzar), imatinib mesylate (Gleevec), irinotecan (Camptosar), methotrexate (Folex, Mexate, Amethopterin), paclitaxel (Taxol, Abraxane), sorafinib (Nexavar), sunitinib (Sutent), topotecan (Hycamtin), vincristine (Oncovin, Vincasar PFS), and vinblastine (Velban).

### VIII. Additional Anti-Cancer Agents

In some embodiments, an additional anti-cancer agent is administered in addition to the treatments otherwise described (e.g., in addition to an immune checkpoint inhibitor and a chemotherapy regimen as described in Section VII or in addition to an ICPI as described in Section VI). It is understood that if a tumor is determined to be a suitable candidate for an ICPI therapy without a chemotherapy regimen using the methods described herein, such as if the tumor is found to have a TMB score of at or higher than 10 mut/megabase or at or higher than 20 mut/megabase, the subject having the tumor may, in some embodiments, further benefit from treatment with an additional anti-cancer agent in addition to the ICPI therapy. Likewise, if a tumor is determined to be a suitable candidate for an immune checkpoint inhibitor with a chemotherapy regimen, such as if the tumor is found to have a TMB score less than 10 mut/megabase, the subject having the tumor may, in some embodiments, further benefit from treatment with an additional anti-cancer agent in addition to the immune checkpoint inhibitor and the chemotherapy regimen.

In some embodiments, the additional anti-cancer therapy comprises an anti-angiogenic agent. In some embodiments, the methods provided herein comprise administering to the individual an anti-angiogenic agent, e.g., in combination with another therapy such as an immune checkpoint inhibitor. Angiogenesis inhibitors prevent the extensive growth of blood vessels (angiogenesis) that tumors require to survive. Non-limiting examples of angiogenesis-mediating molecules or angiogenesis inhibitors which may be used in the methods of the present disclosure include soluble VEGF (for example: VEGF isoforms, e.g., VEGF121 and VEGF165; VEGF receptors, e.g., VEGFR1, VEGFR2; and co-receptors, e.g., Neuropilin-1 and Neuropilin-2), NRP-1, angiopoietin 2, TSP-1 and TSP-2, angiostatin and related molecules, endostatin, vasostatin, calreticulin, platelet factor-4, TIMP and CDAI, Meth-1 and Meth-2, IFNα, IFN-β and IFN-γ, CXCL10, IL-4, IL-12 and IL-18, prothrombin (kringle domain-2), antithrombin III fragment, prolactin, VEGI, SPARC, osteopontin, maspin, canstatin, proliferin-related protein, restin and drugs such as bevacizumab, itraconazole, carboxyamidotriazole, TNP-470, CM101, IFN-a platelet factor-4, suramin, SU5416, thrombospondin, VEGFR antagonists, angiostatic steroids and heparin, cartilage-derived angiogenesis inhibitory factor, matrix metalloproteinase inhibitors, 2-methoxyestradiol, tecogalan, tetrathiomolybdate, thalidomide, thrombospondin, prolactina v β3 inhibitors, linomide, or tasquinimod. In some embodiments, known therapeutic candidates that may be used according to the methods of the disclosure include naturally occurring angiogenic inhibitors, including without limitation, angiostatin, endostatin, or platelet factor-4. In another embodiment, therapeutic candidates that may be used according to the methods of the disclosure include, without limitation, specific inhibitors of endothelial cell growth, such as TNP-470, thalidomide, and interleukin-12. Still other anti-angiogenic agents that may be used according to the methods of the disclosure include those that neutralize angiogenic molecules, including without limitation, antibodies to fibroblast growth factor, antibodies to vascular endothelial growth factor, antibodies to platelet derived growth factor, or antibodies or other types of inhibitors of the receptors of EGF, VEGF or PDGF. In some embodiments, anti-angiogenic agents that may be used according to the methods of the disclosure include, without limitation, suramin and its analogs, and tecogalan. In other embodiments, anti-angiogenic agents that may be used according to the methods of the disclosure include, without limitation, agents that neutralize receptors for angiogenic factors or agents that interfere with vascular basement membrane and extracellular matrix, including, without limitation, metalloprotease inhibitors and angiostatic steroids. Another group of anti-angiogenic compounds that may be used according to the methods of the disclosure includes, without limitation, anti-adhesion molecules, such as antibodies to integrin alpha v beta 3. Still other anti-angiogenic compounds or compositions that may be used according to the methods of the disclosure include, without limitation, kinase inhibitors, thalidomide, itraconazole, carboxyamidotriazole, CM101, IFN-α, IL-12, SU5416, thrombospondin, cartilage-derived angiogenesis inhibitory factor, 2-methoxyestradiol, tetrathiomolybdate, thrombospondin, prolactin, and linomide. In one particular embodiment, the anti-angiogenic compound that may be used according to the methods of the disclosure is an antibody to VEGF, such as Avastin®/bevacizumab (Genentech).

In some embodiments, the additional anti-cancer therapy comprises an anti-DNA repair therapy. In some embodiments, the methods provided herein comprise administering to the individual an anti-DNA repair therapy, e.g., in combination with another therapy such as an immune checkpoint inhibitor. In some embodiments, the anti-DNA repair therapy is a PARP inhibitor (e.g., talazoparib, rucaparib, olaparib), a RAD51 inhibitor (e.g., RI-1), or an inhibitor of a DNA damage response kinase, e.g., CHCK1 (e.g., AZD7762), ATM (e.g., KU-55933, KU-60019, NU7026, or VE-821), and ATR (e.g., NU7026).

In some embodiments, the additional anti-cancer therapy comprises a radiosensitizer. In some embodiments, the methods provided herein comprise administering to the individual a radiosensitizer, e.g., in combination with another therapy such as an immune checkpoint inhibitor. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end-joining (NHEJ), and direct repair mechanisms. Single strand break (SSB) repair mechanisms include BER, NER, or MMR pathways, while double stranded break (DSB) repair mechanisms consist of HR and NHEJ pathways. Radiation causes DNA breaks that, if not repaired, are lethal. SSBs are repaired through a combination of BER, NER and MMR mechanisms using the intact DNA strand as a template. The predominant pathway of SSB repair is BER, utilizing a family of related enzymes termed poly-(ADP-ribose) polymerases (PARP). Thus, the radiosensitizer can include DNA damage response inhibitors such as PARP inhibitors.

In some embodiments, the additional anti-cancer therapy comprises an anti-inflammatory agent. In some embodiments, the methods provided herein comprise administering to the individual an anti-inflammatory agent, e.g., in combination with another therapy such as an immune checkpoint inhibitor. In some embodiments, the anti-inflammatory agent is an agent that blocks, inhibits, or reduces inflammation or signaling from an inflammatory signaling pathway In some embodiments, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23; interferons (IFNs), e.g., IFNα, IFNβ, IFNγ, IFN-γ inducing factor (IGIF); transforming growth factor-β (TGF-β); transforming growth factor-α (TGF-α); tumor necrosis factors, e.g., TNF-α, TNF-β, TNF-RI, TNF-RII; CD23; CD30; CD40L; EGF; G-CSF; GDNF; PDGF-BB; RANTES/CCL5; IKK; NF-κB; TLR2; TLR3; TLR4; TL5; TLR6; TLR7; TLR8; TLR8; TLR9; and/or any cognate receptors thereof. In some embodiments, the anti-inflammatory agent is an IL-1 or IL-1 receptor antagonist, such as anakinra (Kineret®), rilonacept, or canakinumab. In some embodiments, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061. In some embodiments, the anti-inflammatory agent is a TNF-α antagonist, e.g., an anti-TNFα antibody, such as infliximab (Remicade®), golimumab (Simponi®), adalimumab (Humira®), certolizumab pegol (Cimzia®) or etanercept. In some embodiments, the anti-inflammatory agent is a corticosteroid. Exemplary corticosteroids include, but are not limited to, cortisone (hydrocortisone, hydrocortisone sodium phosphate, hydrocortisone sodium succinate, Ala-Cort®, Hydrocort Acetate®, hydrocortone phosphate Lanacort®, Solu-Cortef®), decadron (dexamethasone, dexamethasone acetate, dexamethasone sodium phosphate, Dexasone®, Diodex®, Hexadrol®, Maxidex®), methylprednisolone (6-methylprednisolone, methylprednisolone acetate, methylprednisolone sodium succinate, Duralone®, Medralone®, Medrol®, M-Prednisol®, Solu-Medrol®), prednisolone (Delta-Cortef®, ORAPRED®, Pediapred®, Prezone®), and prednisone (Deltasone®, Liquid Pred®, Meticorten®, Orasone®), and bisphosphonates (e.g., pamidronate (Aredia®), and zoledronic acid (Zometac®).

In some embodiments, the additional anti-cancer therapy comprises an anti-hormonal agent. In some embodiments, the methods provided herein comprise administering to the individual an anti-hormonal agent, e.g., in combination with another therapy such as an immune checkpoint inhibitor. Anti-hormonal agents are agents that act to regulate or inhibit hormone action on tumors. Examples of anti-hormonal agents include anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4 (5)-imidazoles, aminoglutethimide, MEGACE® megestrol acetate, AROMASIN® exemestane, formestanie, fadrozole, RIVISOR® vorozole, FEMARA® letrozole, and ARIMIDEX® (anastrozole); anti-androgens such as flutamide, nilutamide, bicalutamide, leuprolide, and goserelin; troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

In some embodiments, the additional anti-cancer therapy comprises a cancer immunotherapy, such as a cancer vaccine, cell-based therapy, T cell receptor (TCR)-based therapy, adjuvant immunotherapy, cytokine immunotherapy, and oncolytic virus therapy. In some embodiments, the methods provided herein comprise administering to the individual a cancer immunotherapy, such as a cancer vaccine, cell-based therapy, T cell receptor (TCR)-based therapy, adjuvant immunotherapy, cytokine immunotherapy, and oncolytic virus therapy, e.g., in combination with another therapy such as an immune checkpoint inhibitor. In some embodiments, the cancer immunotherapy comprises a small molecule, nucleic acid, polypeptide, carbohydrate, toxin, cell-based agent, or cell-binding agent. Examples of cancer immunotherapies are described in greater detail herein but are not intended to be limiting. In some embodiments, the cancer immunotherapy activates one or more aspects of the immune system to attack a cell (e.g., a tumor cell) that expresses a neoantigen, e.g., a neoantigen expressed by a cancer of the disclosure. The cancer immunotherapies of the present disclosure are contemplated for use as monotherapies, or in combination approaches comprising two or more in any combination or number, subject to medical judgement. Any of the cancer immunotherapies (optionally as monotherapies or in combination with another cancer immunotherapy or other therapeutic agent described herein) may find use in any of the methods described herein.

In some embodiments, the cancer immunotherapy comprises a cancer vaccine. A range of cancer vaccines have been tested that employ different approaches to promoting an immune response against a cancer (see, e.g., Emens L A, Expert Opin Emerg Drugs 13 (2): 295-308 (2008) and US20190367613). Approaches have been designed to enhance the response of B cells, T cells, or professional antigen-presenting cells against tumors. Exemplary types of cancer vaccines include, but are not limited to, DNA-based vaccines, RNA-based vaccines, virus transduced vaccines, peptide-based vaccines, dendritic cell vaccines, oncolytic viruses, whole tumor cell vaccines, tumor antigen vaccines, etc. In some embodiments, the cancer vaccine can be prophylactic or therapeutic. In some embodiments, the cancer vaccine is formulated as a peptide-based vaccine, a nucleic acid-based vaccine, an antibody-based vaccine, or a cell-based vaccine. For example, a vaccine composition can include naked cDNA in cationic lipid formulations; lipopeptides (e.g., Vitiello, A. et ah, J. Clin. Invest. 95:341, 1995), naked cDNA or peptides, encapsulated e.g., in poly(DL-lactide-co-glycolide) (“PLG”) microspheres (see, e.g., Eldridge, et ah, Molec. Immunol. 28:287-294, 1991: Alonso et al, Vaccine 12:299-306, 1994; Jones et al, Vaccine 13:675-681, 1995); peptide composition contained in immune stimulating complexes (ISCOMS) (e.g., Takahashi et al, Nature 344:873-875, 1990; Hu et al, Clin. Exp. Immunol. 113:235-243, 1998); or multiple antigen peptide systems (MAPs) (see e.g., Tam, J. P., Proc. Natl Acad. Sci. U.S.A. 85:5409-5413, 1988; Tam, J. P., J. Immunol. Methods 196:17-32, 1996). In some embodiments, a cancer vaccine is formulated as a peptide-based vaccine, or nucleic acid-based vaccine in which the nucleic acid encodes the polypeptides. In some embodiments, a cancer vaccine is formulated as an antibody-based vaccine. In some embodiments, a cancer vaccine is formulated as a cell-based vaccine. In some embodiments, the cancer vaccine is a peptide cancer vaccine, which in some embodiments is a personalized peptide vaccine. In some embodiments, the cancer vaccine is a multivalent long peptide, a multiple peptide, a peptide mixture, a hybrid peptide, or a peptide pulsed dendritic cell vaccine (see, e.g., Yamada et al, Cancer Sci, 104:14-21), 2013). In some embodiments, such cancer vaccines augment the anti-cancer response.

In some embodiments, the cancer vaccine comprises a polynucleotide that encodes a neoantigen, e.g., a neoantigen expressed by a cancer of the disclosure. In some embodiments, the cancer vaccine comprises DNA or RNA that encodes a neoantigen. In some embodiments, the cancer vaccine comprises a polynucleotide that encodes a neoantigen. In some embodiments, the cancer vaccine further comprises one or more additional antigens, neoantigens, or other sequences that promote antigen presentation and/or an immune response. In some embodiments, the polynucleotide is complexed with one or more additional agents, such as a liposome or lipoplex. In some embodiments, the polynucleotide(s) are taken up and translated by antigen presenting cells (APCs), which then present the neoantigen(s) via MHC class I on the APC cell surface.

In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, the cancer vaccine is selected from an oncolytic viral therapy such as pexastimogene devacirepvec (PexaVec/JX-594, SillaJen/formerly Jennerex Biotherapeutics), a thymidine kinase-(TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543), prostate cancer (NCT01619813), head and neck squamous cell cancer (NCT01166542), pancreatic adenocarcinoma (NCT00998322), and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117), metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676), and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260), fallopian tube cancer, ovarian cancer (NCT 02759588); or CG0070 (Cold Genesys), an adenovirus engineered to express GM-CSF in bladder cancer (NCT02365818); anti-gp100; STINGVAX; GVAX; DCVaxL; and DNX-2401. In some embodiments, the cancer vaccine is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TGO1 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFa-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8+ T cell response. In some embodiments, the cancer vaccine comprises a vector-based tumor antigen vaccine. Vector-based tumor antigen vaccines can be used as a way to provide a steady supply of antigens to stimulate an anti-tumor immune response. In some embodiments, vectors encoding for tumor antigens are injected into an individual (possibly with pro-inflammatory or other attractants such as GM-CSF), taken up by cells in vivo to make the specific antigens, which then provoke the desired immune response. In some embodiments, vectors may be used to deliver more than one tumor antigen at a time, to increase the immune response. In addition, recombinant virus, bacteria, or yeast vectors can trigger their own immune responses, which may also enhance the overall immune response.

In some embodiments, the cancer vaccine comprises a DNA-based vaccine. In some embodiments, DNA-based vaccines can be employed to stimulate an anti-tumor response. The ability of directly injected DNA that encodes an antigenic protein, to elicit a protective immune response has been demonstrated in numerous experimental systems. Vaccination through directly injecting DNA that encodes an antigenic protein, to elicit a protective immune response often produces both cell-mediated and humoral responses. Moreover, reproducible immune responses to DNA encoding various antigens have been reported in mice that last essentially for the lifetime of the animal (see, e.g., Yankauckas et al. (1993) DNA Cell Biol., 12:771-776). In some embodiments, plasmid (or other vector) DNA that includes a sequence encoding a protein operably linked to regulatory elements required for gene expression is administered to individuals (e.g., human patients, non-human mammals, etc.). In some embodiments, the cells of the individual take up the administered DNA and the coding sequence is expressed. In some embodiments, the antigen so produced becomes a target against which an immune response is directed.

In some embodiments, the cancer vaccine comprises an RNA-based vaccine. In some embodiments, RNA-based vaccines can be employed to stimulate an anti-tumor response. In some embodiments, RNA-based vaccines comprise a self-replicating RNA molecule. In some embodiments, the self-replicating RNA molecule may be an alphavirus-derived RNA replicon. Self-replicating RNA (or “SAM”) molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides an RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen.

In some embodiments, the cancer immunotherapy comprises a cell-based therapy. In some embodiments, the cancer immunotherapy comprises a T cell-based therapy. In some embodiments, the cancer immunotherapy comprises an adoptive therapy, e.g., an adoptive T cell-based therapy. In some embodiments, the T cells are autologous or allogeneic to the recipient. In some embodiments, the T cells are CD8+ T cells. In some embodiments, the T cells are CD4+ T cells. Adoptive immunotherapy refers to a therapeutic approach for treating cancer or infectious diseases in which immune cells are administered to a host with the aim that the cells mediate either directly or indirectly specific immunity to (i.e., mount an immune response directed against) cancer cells. In some embodiments, the immune response results in inhibition of tumor and/or metastatic cell growth and/or proliferation, and in related embodiments, results in neoplastic cell death and/or resorption. The immune cells can be derived from a different organism/host (exogenous immune cells) or can be cells obtained from the subject organism (autologous immune cells). In some embodiments, the immune cells (e.g., autologous or allogeneic T cells (e.g., regulatory T cells, CD4+ T cells, CD8+ T cells, or gamma-delta T cells), NK cells, invariant NK cells, or NKT cells) can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In some embodiments, NK cells are engineered to express a TCR. The NK cells may be further engineered to express a CAR. Multiple CARs and/or TCRs, such as to different antigens, may be added to a single cell type, such as T cells or NK cells. In some embodiments, the cells comprise one or more nucleic acids/expression constructs/vectors introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric). In some embodiments, a population of immune cells can be obtained from a subject in need of therapy or suffering from a disease associated with reduced immune cell activity. Thus, the cells will be autologous to the subject in need of therapy. In some embodiments, a population of immune cells can be obtained from a donor, such as a histocompatibility-matched donor. In some embodiments, the immune cell population can be harvested from the peripheral blood, cord blood, bone marrow, spleen, or any other organ/tissue in which immune cells reside in said subject or donor. In some embodiments, the immune cells can be isolated from a pool of subjects and/or donors, such as from pooled cord blood. In some embodiments, when the population of immune cells is obtained from a donor distinct from the subject, the donor may be allogeneic, provided the cells obtained are subject-compatible, in that they can be introduced into the subject. In some embodiments, allogeneic donor cells may or may not be human-leukocyte-antigen (HLA)-compatible. In some embodiments, to be rendered subject-compatible, allogeneic cells can be treated to reduce immunogenicity.

In some embodiments, the cell-based therapy comprises a T cell-based therapy, such as autologous cells, e.g., tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. Several approaches for the isolation, derivation, engineering or modification, activation, and expansion of functional anti-tumor effector cells have been described in the last two decades and may be used according to any of the methods provided herein. In some embodiments, the T cells are derived from the blood, bone marrow, lymph, umbilical cord, or lymphoid organs. In some embodiments, the cells are human cells. In some embodiments, the cells are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells may be allogeneic and/or autologous. In some embodiments, such as for off-the-shelf technologies, the cells are pluripotent and/or multipotent, such as stem cells, such as induced pluripotent stem cells (iPSCs).

In some embodiments, the T cell-based therapy comprises a chimeric antigen receptor (CAR)-T cell-based therapy. This approach involves engineering a CAR that specifically binds to an antigen of interest and comprises one or more intracellular signaling domains for T cell activation. The CAR is then expressed on the surface of engineered T cells (CAR-T) and administered to a patient, leading to a T-cell-specific immune response against cancer cells expressing the antigen.

In some embodiments, the T cell-based therapy comprises T cells expressing a recombinant T cell receptor (TCR). This approach involves identifying a TCR that specifically binds to an antigen of interest, which is then used to replace the endogenous or native TCR on the surface of engineered T cells that are administered to a patient, leading to a T-cell-specific immune response against cancer cells expressing the antigen.

In some embodiments, the T cell-based therapy comprises tumor-infiltrating lymphocytes (TILs). For example, TILs can be isolated from a tumor or cancer of the present disclosure, then isolated and expanded in vitro. Some or all of these TILs may specifically recognize an antigen expressed by the tumor or cancer of the present disclosure. In some embodiments, the TILs are exposed to one or more neoantigens, e.g., a neoantigen, in vitro after isolation. TILs are then administered to the patient (optionally in combination with one or more cytokines or other immune-stimulating substances).

In some embodiments, the cell-based therapy comprises a natural killer (NK) cell-based therapy. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells can be detected by specific surface markers, such as CD16, CD56, and CD8 in humans. NK cells do not express T-cell antigen receptors, the pan T marker CD3, or surface immunoglobulin B cell receptors. In some embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art.

In some embodiments, the cell-based therapy comprises a dendritic cell (DC)-based therapy, e.g., a dendritic cell vaccine. In some embodiments, the DC vaccine comprises antigen-presenting cells that are able to induce specific T cell immunity, which are harvested from the patient or from a donor. In some embodiments, the DC vaccine can then be exposed in vitro to a peptide antigen, for which T cells are to be generated in the patient. In some embodiments, dendritic cells loaded with the antigen are then injected back into the patient. In some embodiments, immunization may be repeated multiple times if desired. Methods for harvesting, expanding, and administering dendritic cells are known in the art; see, e.g., U.S. patent application No. 20210213058, the content of which is herein incorporated by reference in its entirety. Dendritic cell vaccines (such as Sipuleucel-T, also known as APC8015 and PROVENGER) are vaccines that involve administration of dendritic cells that act as APCs to present one or more cancer-specific antigens to the patient's immune system. In some embodiments, the dendritic cells are autologous or allogeneic to the recipient.

In some embodiments, the cancer immunotherapy comprises a TCR-based therapy. In some embodiments, the cancer immunotherapy comprises administration of one or more TCRs or TCR-based therapeutics that specifically bind an antigen expressed by a cancer of the present disclosure. In some embodiments, the TCR-based therapeutic may further include a moiety that binds an immune cell (e.g., a T cell), such as an antibody or antibody fragment that specifically binds a T cell surface protein or receptor (e.g., an anti-CD3 antibody or antibody fragment).

In some embodiments, the immunotherapy comprises adjuvant immunotherapy. Adjuvant immunotherapy comprises the use of one or more agents that activate components of the innate immune system, e.g., HILTONOL® (imiquimod), which targets the TLR7 pathway.

In some embodiments, the immunotherapy comprises cytokine immunotherapy. Cytokine immunotherapy comprises the use of one or more cytokines that activate components of the immune system. Examples include, but are not limited to, aldesleukin (PROLEUKIN®; interleukin-2), interferon alfa-2a (ROFERON®-A), interferon alfa-2b (INTRON®-A), and peginterferon alfa-2b (PEGINTRON®).

In some embodiments, the immunotherapy comprises oncolytic virus therapy. Oncolytic virus therapy uses genetically modified viruses to replicate in and kill cancer cells, leading to the release of antigens that stimulate an immune response. In some embodiments, replication-competent oncolytic viruses expressing a tumor antigen comprise any naturally occurring (e.g., from a “field source”) or modified replication-competent oncolytic virus. In some embodiments, the oncolytic virus, in addition to expressing a tumor antigen, may be modified to increase selectivity of the virus for cancer cells. In some embodiments, replication-competent oncolytic viruses include, but are not limited to, oncolytic viruses that are a member in the family of myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hcpadnaviridae, retroviridae, cyctoviridae, reoviridae, birnaviridae, paramyxoviridae, rhabdoviridae, filoviridae, orthomyxoviridae, bunyaviridae, arenaviridae, Leviviridae, picornaviridae, sequiviridae, comoviridae, potyviridae, caliciviridae, astroviridae, nodaviridae, tetraviridae, tombusviridae, coronaviridae, glaviviridae, togaviridae, and barnaviridae. In some embodiments, replication-competent oncolytic viruses include adenovirus, retrovirus, reovirus, rhabdovirus, Newcastle Disease virus (NDV), polyoma virus, vaccinia virus (VacV), herpes simplex virus, picornavirus, coxsackie virus and parvovirus. In some embodiments, a replicative oncolytic vaccinia virus expressing a tumor antigen may be engineered to lack one or more functional genes in order to increase the cancer selectivity of the virus. In some embodiments, an oncolytic vaccinia virus is engineered to lack thymidine kinase (TK) activity. In some embodiments, the oncolytic vaccinia virus may be engineered to lack vaccinia virus growth factor (VGF). In some embodiments, an oncolytic vaccinia virus may be engineered to lack both VGF and TK activity. In some embodiments, an oncolytic vaccinia virus may be engineered to lack one or more genes involved in evading host interferon (IFN) response such as E3L, K3L, B18R, or B8R. In some embodiments, a replicative oncolytic vaccinia virus is a Western Reserve, Copenhagen, Lister or Wyeth strain and lacks a functional TK gene. In some embodiments, the oncolytic vaccinia virus is a Western Reserve, Copenhagen, Lister or Wyeth strain lacking a functional B18R and/or B8R gene. In some embodiments, a replicative oncolytic vaccinia virus expressing a tumor antigen may be locally or systemically administered to a subject, e.g., via intratumoral, intraperitoneal, intravenous, intra-arterial, intramuscular, intradermal, intracranial, subcutaneous, or intranasal administration.

In some embodiments, the anti-cancer therapy comprises a nucleic acid molecule, such as a dsRNA, an siRNA, or an shRNA. In some embodiments, the methods provided herein comprise administering to the individual a nucleic acid molecule, such as a dsRNA, an siRNA, or an shRNA, e.g., in combination with another anti-cancer therapy. As is known in the art, dsRNAs having a duplex structure are effective at inducing RNA interference (RNAi). In some embodiments, the anti-cancer therapy comprises a small interfering RNA molecule (siRNA). dsRNAs and siRNAs can be used to silence gene expression in mammalian cells (e.g., human cells). In some embodiments, a dsRNA of the disclosure comprises any of between about 5 and about 10 base pairs, between about 10 and about 12 base pairs, between about 12 and about 15 base pairs, between about 15 and about 20 base pairs, between about 20 and 23 base pairs, between about 23 and about 25 base pairs, between about 25 and about 27 base pairs, or between about 27 and about 30 base pairs. As is known in the art, siRNAs are small dsRNAs that optionally include overhangs. In some embodiments, the duplex region of an siRNA is between about 18 and 25 nucleotides, e.g., any of 18, 19, 20, 21, 22, 23, 24, or 25 nucleotides. siRNAs may also include short hairpin RNAs (shRNAs), e.g., with approximately 29-base-pair stems and 2-nucleotide 3′ overhangs. Methods for designing, optimizing, producing, and using dsRNAs, siRNAs, or shRNAs, are known in the art.

### IX. Treatments and Treatment Effects

The methods described herein provide improved therapies and/or therapeutic effects. The improved therapies and/or therapeutic effects are based, in part, on the stratification of individuals with gastroesophageal cancer having a TMB score below a threshold TMB score and individuals with gastroesophageal cancer having a TMB score at or higher than a threshold TMB score. Once a TMB score is determined, the individuals can receive appropriate therapies, leading to improved clinical outcomes including improved survival (such as improved progression-free survival and/or improved overall survival) and/or increased time to next treatment (TTNT). The individuals may be any of the individuals described in Section III, above.

Accordingly, in some embodiments, the methods comprise administering an immune checkpoint inhibitor (ICPI, such as an ICPI described in Section VI) without a chemotherapy regimen if a TMB score in a sample from an individual having a gastroesophageal cancer is at or higher than a threshold TMB score. In some embodiments, the methods comprise administering an immune checkpoint inhibitor therapy with a chemotherapy (such as a chemotherapy described in Section VII) if a TMB score is below a threshold TMB score.

In some embodiments, the methods of treatment described herein provide a clinical benefit and/or an improved clinical benefit for individuals having a gastroesophageal cancer. In some embodiments, the methods provide improved clinical benefit when compared to an alternative therapy. For example, in some embodiments, the methods comprise administering an ICPI without a chemotherapy regimen, wherein the individual will, or is expected to, benefit from the ICPI therapy alone as compared to treatment with an ICPI with a chemotherapy regimen. In some embodiments, the methods comprise administering an ICPI therapy and a chemotherapy regimen, where the individual will, or is expected to, benefit from the ICPI therapy and a chemotherapy regimen as compared to treatment with an ICPI therapy alone. In some embodiments, the methods comprise administering an ICPI without a chemotherapy regimen, wherein the individual will, or is expected to, have a comparable benefit from the ICPI therapy alone as compared to treatment with an ICPI therapy with a chemotherapy regimen. It is contemplated that treatment of an individual with an ICPI therapy alone has certain advantages over treatment with a combination therapy comprising an ICPI and a chemotherapy regimen including, but not limited to, reduced toxicity from cancer treatment as well as reduced ancillary hospital costs associated with chemotherapy use (e.g., costs from infusions, ER visits, etc.).

In some embodiments, the clinical benefit is improved survival (such as improved PFS and/or improved OS). In some embodiments, the treatment improves PFS by at least one month, such as any of at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 18 months, at least about 2 years, at least about 3 years, at least about 4 years, or more, after administration. In some embodiments, the treatment improves OS by at least one month, such as any of at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 18 months, at least about 2 years, at least about 3 years, at least about 4 years, or more, after administration. In some embodiments, the treatment methods provide improved objective response rate of at least 20%, such as any of about 25%, about 30%, about 35%, about 40%, about 45%, about 50%, about 55%, about 60%, about 65%, about 70%, about 75%, about 80%, about 85%, about 90%, about 95%, or about 100%.

### X. Cancers to be Assessed or Treated

The methods described herein pertain to individuals having a gastroesophageal cancer and assessment of the cancers (by assessment of a sample, such as a blood sample or a tumor biopsy sample) to identify suitable treatments for the individual. In some embodiments, the cancer is gastroesophageal cancer. In some embodiments, the cancer is advanced gastroesophageal cancer. In some embodiments, the cancer is metastatic gastroesophageal cancer. Premaligancy, as used herein, refers to a tissue that is not yet malignant but is poised to become malignant.

In some embodiments, the gastroesophageal cancer to be treated or assessed has never been treated with an anti-cancer therapy. In some embodiments, the gastroesophageal cancer to be treated or assessed has never been treated, or is not currently being treated, with a chemotherapy regimen.

In some embodiments, the gastroesophageal cancer to be treated or assessed has a TMB score of at or higher than 8 mutations/Mb, such as any of about 9 mutations/Mb, about 10 mutations/Mb, about 11 mutations/Mb, about 12 mutations/Mb, about 13 mutations/Mb, about 14 mutations/Mb, about 15 mutations/Mb, about 16 mutations/Mb, about 17 mutations/Mb, about 18 mutations/Mb, about 19 mutations/Mb, about 20 mutations/Mb, or more. In some embodiments, the gastroesophageal cancer to be treated or assessed has a TMB score of at or higher than 10 mutations/Mb. In some embodiments, the gastroesophageal cancer to be treated or assessed has a TMB score of less than 12 mutations/Mb, such as any of less than about 11 mutations/Mb, about 10 mutations/Mb, about 9 mutations/Mb, about 8 mutations/Mb, about 7 mutations/Mb, about 6 mutations/Mb, or less. In some embodiments, the gastroesophageal cancer to be treated or assessed has a TMB score of less than 10 mutations/Mb.

### XI. Exemplary Embodiments

The following embodiments are exemplary and are not intended to limit the scope of the invention.

Embodiment 1. A method for treating an individual having a gastroesophageal cancer, the method comprising:
(a) determining a tumor mutational burden (TMB) score for a biopsy sample obtained from the individual; and
(b) administering an immune checkpoint inhibitor without a chemotherapy regimen to the individual if the TMB score is at or higher than a threshold TMB score.
   Embodiment 2. A method for identifying an individual having a gastroesophageal cancer for treatment with an immune checkpoint inhibitor therapy without a chemotherapy regimen comprising determining a tumor mutational burden (TMB) score for a biopsy sample obtained from the individual, wherein if the TMB score is at or higher than a threshold TMB score the individual is identified for treatment with an immune checkpoint inhibitor therapy without a chemotherapy regimen.
   Embodiment 3. A method of selecting a treatment for an individual having a gastroesophageal cancer, the method comprising determining a tumor mutational burden (TMB) score for a biopsy sample obtained from the individual, wherein a TMB score that is at or higher than a threshold TMB score identifies the individual as one who may benefit from treatment with an immune checkpoint inhibitor therapy without a chemotherapy regimen.
   Embodiment 4. A method of identifying one or more treatment options for an individual having a gastroesophageal cancer, the method comprising:
(a) determining a tumor mutational burden (TMB) score for a biopsy sample obtained from the individual; and
(b) generating a report comprising one or more treatment options identified for the individual, wherein a TMB score that is at or higher than a threshold TMB score identifies the individual as one who may benefit from treatment with an immune checkpoint inhibitor therapy without a chemotherapy regimen.
   Embodiment 5. A method of stratifying an individual with a gastroesophageal cancer for treatment with a therapy comprising determining a tumor mutational burden (TMB) score for a biopsy sample obtained from the individual; and
(a) if the TMB score is at or higher than a threshold TMB score, identifying the individual as a candidate for receiving an immune checkpoint inhibitor therapy without a chemotherapy regimen, or
(b) if the TMB score is less than a threshold TMB score, identifying the individual as a candidate for receiving an immune checkpoint inhibitor therapy and a chemotherapy regimen.
   Embodiment 6. A method of predicting survival of an individual having a gastroesophageal cancer, comprising acquiring knowledge of a tumor mutational burden (TMB) score for a biopsy sample obtained from the individual, wherein if the TMB score for the biopsy sample obtained from the individual is at or higher than a threshold TMB score, the individual is predicted to have increased survival when treated with an immune checkpoint inhibitor without a chemotherapy regimen, as compared to treatment with an immune checkpoint inhibitor therapy and a chemotherapy regimen.
   Embodiment 7. A method of monitoring, evaluating, or screening an individual having a gastroesophageal cancer, comprising acquiring knowledge of a tumor mutational burden (TMB) score for a biopsy sample obtained from the individual, wherein if the TMB score for the biopsy sample obtained from the individual is at or higher than a threshold TMB score, the individual is predicted to have increased survival when treated with an immune checkpoint inhibitor without a chemotherapy regimen, as compared to treatment with an immune checkpoint inhibitor therapy and a chemotherapy regimen.
   Embodiment 8. The method of any one of embodiments 1-7, wherein the threshold TMB score is about 8 mutations/Mb, about 9 mutations/Mb, about 10 mutations/Mb, about 11 mutations/Mb, about 12 mutations/Mb, about 13 mutations/Mb, about 14 mutations/Mb, about 15 mutations/Mb, about 16 mutations/Mb, about 17 mutations/Mb, about 18 mutations/Mb, about 19 mutations/Mb, or about 20 mutations/Mb.
   Embodiment 9. The method of embodiment 8, wherein the threshold TMB score is about 10 mutations/Mb.
   Embodiment 10. The method of any one of embodiments 1-9, wherein the TMB score is determined based on between about 100 kb to about 10 Mb of sequenced DNA.
   Embodiment 11. The method of embodiment 10, wherein the TMB score is determined based on between about 0.8 Mb to about 1.1 Mb of sequenced DNA.
   Embodiment 12. The method of any one of embodiments 2-11, further comprising administering an immune checkpoint inhibitor without a chemotherapy regimen to the individual if the TMB score is at or higher than the threshold TMB score.
   Embodiment 13. The method of any one of embodiments 1-12, further comprising administering an immune checkpoint inhibitor and a chemotherapy regimen to the individual if the TMB score is less than the threshold TMB score.
   Embodiment 14. The method of any one of embodiments 1-13, wherein the immune checkpoint inhibitor therapy is a first line therapy for the gastroesophageal cancer.
   Embodiment 15. The method of any one of embodiments 1-14, wherein the immune checkpoint inhibitor comprises a small molecule inhibitor, an antibody, a nucleic acid, an antibody-drug conjugate, a recombinant protein, a fusion protein, a natural compound, a peptide, a PROteolysis-TArgeting Chimera (PROTAC), a cellular therapy, a treatment for cancer being tested in a clinical trial, an immunotherapy, or any combination thereof.
   Embodiment 16. The method of embodiment 15, wherein the immune checkpoint inhibitor is a PD-1 inhibitor.
   Embodiment 17. The method of embodiment 16, wherein the PD-1 inhibitor comprises one or more of nivolumab, pembrolizumab, cemiplimab, or dostarlimab.
   Embodiment 18. The method of embodiment 15, wherein the immune checkpoint inhibitor is a PD-L1 inhibitor.
   Embodiment 19. The method of embodiment 18, wherein the PD-L1 inhibitor comprises one or more of atezolizumab, avelumab, or durvalumab.
   Embodiment 20. The method of embodiment 15, wherein the immune checkpoint inhibitor is a CTLA-4 inhibitor.
   Embodiment 21. The method of embodiment 20, wherein the CTLA-4 inhibitor comprises ipilimumab.
   Embodiment 22. The method of any one of embodiments 1-21, wherein the immune checkpoint inhibitor therapy is the only anti-cancer therapy indicated or administered for the gastroesophageal cancer.
   Embodiment 23. The method of any one of embodiments 1-22, wherein the immune checkpoint inhibitor therapy is a single-active-agent therapy.
   Embodiment 24. The method of any one of embodiments 1-22, wherein the immune checkpoint inhibitor therapy comprises two or more active agents.
   Embodiment 25. The method of any one of embodiments 1-24, wherein the immune checkpoint inhibitor therapy comprises a first round of an immune checkpoint inhibitor and a subsequent round of therapy with a different immune checkpoint inhibitor.
   Embodiment 26. The method of any one of embodiments 5-25, wherein the chemotherapy comprises one or more of an alkylating agent, an alkyl sulfonates aziridine, an ethylenimine, a methylamelamine, an acetogenin, a camptothecin, a bryostatin, a callystatin, CC-1065, a cryptophycin, aa dolastatin, a duocarmycin, a eleutherobin, a pancratistatin, a sarcodictyin, a spongistatin, a nitrogen mustard, a nitrosureas, an antibiotic, a dynemicin, a bisphosphonate, an esperamicina a neocarzinostatin chromophore or a related chromoprotein enediyne antiobiotic chromophore, an anti-metabolite, a folic acid analogue, a purine analog, a pyrimidine analog, an androgens, an anti-adrenal, a folic acid replenisher, aldophosphamide glycoside, aminolevulinic acid, eniluracil, amsacrine, bestrabucil, bisantrene, edatraxate, defofamine, demecolcine, diaziquone, elformithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, a PSK polysaccharide complex, razoxane, rhizoxin, sizofiran, spirogermanium, tenuazonic acid, triaziquone, 2,2′,2″-trichlorotriethylamine, a trichothecene, urethan, vindesine, dacarbazine, mannomustine, mitobronitol, mitolactol, pipobroman, gacytosine, arabinoside (“Ara-C”), cyclophosphamide, a taxoid, 6-thioguanine, mercaptopurine, a platinum coordination complex, vinblastine, platinum, etoposide (VP-16), ifosfamide, mitoxantrone, vincristine, vinorelbine, novantrone, teniposide, edatrexate, daunomycin, aminopterin, xeloda, ibandronate, irinotecan, topoisomerase inhibitor RFS 2000, difluorometlhylomithine (DMFO), a retinoid, Capecitabine, carboplatin, procarbazine, plicomycin, gemcitabine, navelbine, a famesyl-protein tansferase inhibitor, transplatinum, fluorouracil, a taxane, or any combination thereof.
   Embodiment 27. The method of any one of embodiments 1-26, further comprising administering an additional anti-cancer therapy to the individual.
   Embodiment 28. The method of embodiment 27, wherein the additional anti-cancer therapy comprises one or more of a small molecule inhibitor, a cancer immunotherapy, an antibody, a cellular therapy, a nucleic acid, a surgery, a radiotherapy, an anti-angiogenic therapy, an anti-DNA repair therapy, an anti-inflammatory therapy, a growth inhibitory agent, or any combination thereof.
   Embodiment 29. The method of any one of embodiments 1-28, wherein the individual did not previously receive a regimen of chemotherapy for the gastroesophageal cancer.
   Embodiment 30. The method of any one of embodiments 1-29, wherein the TMB score is determined by sequencing.
   Embodiment 31. The method of embodiment 30, wherein the sequencing comprises use of a massively parallel sequencing (MPS) technique, whole genome sequencing (WGS), whole exome sequencing (WES), targeted sequencing, direct sequencing, next-generation sequencing (NGS), or a Sanger sequencing technique.
   Embodiment 32. The method of embodiment 30 or embodiment 31, wherein the sequencing comprises:
(a) providing a plurality of nucleic acid molecules obtained from the biopsy sample, wherein the plurality of nucleic acid molecules comprise a mixture of tumor nucleic acid molecules and non-tumor nucleic acid molecules;
(b) optionally, ligating one or more adapters onto one or more nucleic acid molecules from the plurality of nucleic acid molecules;
(c) amplifying nucleic acid molecules from the plurality of nucleic acid molecules;
(d) capturing nucleic acid molecules from the amplified nucleic acid molecules, wherein the captured nucleic acid molecules are captured from the amplified nucleic acid molecules by hybridization to one or more bait molecules; and
(e) sequencing, by a sequencer, at or higher than a portion of the captured nucleic acid molecules to obtain a plurality of sequence reads corresponding to one or more genomic loci within a subgenomic interval in the sample.
   Embodiment 33. The method of embodiment 32, wherein the adapters comprise one or more of amplification primer sequences, flow cell adapter hybridization sequences, unique molecular identifier sequences, substrate adapter sequences, or sample index sequences.
   Embodiment 34. The method of embodiment 32 or embodiment 33, wherein amplyifying nucleic acid molecules comprises performing a polymerase chain reaction (PCR) technique, a non-PCR amplification technique, or an isothermal amplification technique.
   Embodiment 35. The method of any one of embodiments 32-34, wherein the one or more bait molecules comprise one or more nucleic acid molecules, each comprising a region that is complementary to a region of a captured nucleic acid molecule.
   Embodiment 36. The method of embodiment 35, wherein the one or more bait molecules each comprise a capture moiety.
   Embodiment 37. The method of embodiment 36, wherein the capture moiety is biotin.
   Embodiment 38. The method of any one of embodiments 1-5 and 8-37, wherein if the TMB score is at or higher than a threshold TMB score, the individual is identified to have an increased survival when treated with an immune checkpoint inhibitor without a chemotherapy regimen as compared to treatment with an immune checkpoint inhibitor therapy and a chemotherapy regimen.
   Embodiment 39. The method of any one of embodiments 6-38, wherein the increased survival is increased overall survival (OS).
   Embodiment 40. The method of any one of embodiments 6-39, wherein the increased survival is increased progression-free survival (PFS).
   Embodiment 41. The method of any one of embodiments 1-40, wherein the gastroesophageal cancer is advanced.
   Embodiment 42. The method of any one of embodiments 1-41, wherein the gastroesophageal cancer is metastatic.
   Embodiment 43. The method of any one of embodiment 1-42, wherein the biopsy sample is a tumor biopsy sample.
   Embodiment 44. The method of any one of embodiment 1-42, wherein the biopsy sample is a liquid biopsy sample.
   Embodiment 45. The method of any one of embodiments 1-44, wherein the individual is a human.
   Embodiment 46. A kit comprising an immune checkpoint inhibitor and instructions for use according to the method of any one of embodiments 1-45.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: De-Risking 1st Line Chemotherapy-Free Immune Checkpoint Inhibitor (ICI) Use for Patients with Advanced Gastroesophageal Cancer (aGEJ) with Tumor Mutational Burden (TMB)

### BACKGROUND

ICI+chemotherapy (chemo-ICI) is becoming an increasingly common 1st line regimen for patients with aGEJ. However, direct outcome comparison between biomarker-enriched groups receiving chemo-ICI vs. ICI monotherapy (mono-ICI) is limited in phase III trials. A goal of this example was to evaluate if TMB might identify patients for whom mono-ICI vs. chemo-ICI might be de-risked. It was hypothesized that (1) patients with TMB<10 mut/mB would have more favorable outcomes receiving ICI-chemo vs. mono-ICI and (2) patients with TMB≥10 mut/mB would have diminished additional benefit from chemo-ICI vs. mono-ICI.

### Materials and Methods

This study included patients with aGEJ who received 1st line chemo-ICI or mono-ICI and tumor tissue genomic profiling. Patient data was obtained by a nationwide (US-based) de-identified gastric real-world clinico-genomic database, originating from approximately 280 US cancer clinics (January/2011-March/2023). Real-world progression-free survival (rwPFS) and overall survival (rwOS) were compared between patients receiving chemo-ICI vs. mono-ICI by Cox models, adjusted by propensity scores accounting for disease stage at diagnosis, ECOG PS, PD-L1 score and microsatellite instability (MSI) status. Comparisons of the explanatory power of TMB (<10 vs.≥10 mut/mB) vs. MSI status (MSI-H vs MSS) were performed by likelihood ratio test (LRT).

TABLE 1
Characteristics of the overall Patient Cohort

| | | ICI | | |
|---|---|---|---|---|
| | ICI + Chemo | Monotherapy | Total | p |
| | (N = 176) | (N = 87) | (N = 263) | value |
| Age | | | | 0.501 |
| Median (Q1, Q3) | 67.0 (59.8, 73.0) | 66.0 (59.0, 76.5) | 67.0 (59.0, 74.0) | |
| Sex | | | | 0.151 |
| Female | 42 (23.9%) | 28 (32.2%) | 70 (26.6%) | |
| Male | 134 (76.1%) | 59 (67.8%) | 193 (73.4%) | |
| Race | | | | 0.01 |
| Asian | 3 (1.7%) | 7 (8.0%) | 10 (3.8%) | |
| Black or African | 9 (5.1%) | 6 (6.9%) | 15 (5.7%) | |
| American | | | | |
| Hispanic or Latino | 0 (0.0%) | 1 (1.1%) | 1 (0.4%) | |
| Other Race | 24 (13.6%) | 20 (23.0%) | 44 (16.7%) | |
| Unknown/not | 23 (13.1%) | 12 (13.8%) | 35 (13.3%) | |
| documented | | | | |
| White | 117 (66.5%) | 41 (47.1%) | 158 (60.1%) | |
| Practice Type | | | | 0.587 |
| Academic | 11 (6.2%) | 7 (8.0%) | 18 (6.8%) | |
| Community | 165 (93.8%) | 80 (92.0%) | 245 (93.2%) | |
| History of Smoking | | | | 0.15 |
| History of smoking | 126 (71.6%) | 52 (59.8%) | 178 (67.7%) | |
| No history of smoking | 49 (27.8%) | 34 (39.1%) | 83 (31.6%) | |
| Unknown/Not | 1 (0.6%) | 1 (1.1%) | 2 (0.8%) | |
| documented | | | | |
| H Pylori DNA in | | | | 0.481 |
| Tumor | | | | |
| Not detected | 175 (99.4%) | 87 (100.0%) | 262 (99.6%) | |
| Positive | 1 (0.6%) | 0 (0.0%) | 1 (0.4%) | |
| Site of Primary Tumor | | | | 0.075 |
| Esophageal | 94 (53.4%) | 35 (40.2%) | 129 (49.0%) | |
| Gastric | 43 (24.4%) | 32 (36.8%) | 75 (28.5%) | |
| GEJ | 39 (22.2%) | 20 (23.0%) | 59 (22.4%) | |
| Histology | | | | 0.345 |
| Adenocarcinoma | 152 (86.4%) | 68 (78.2%) | 220 (83.7%) | |
| Adenosquamous | 2 (1.1%) | 1 (1.1%) | 3 (1.1%) | |
| Other | 1 (0.6%) | 1 (1.1%) | 2 (0.8%) | |
| Squamous Cell | 21 (11.9%) | 16 (18.4%) | 37 (14.1%) | |
| Carcinoma | | | | |
| Unknown/Not | 0 (0.0%) | 1 (1.1%) | 1 (0.4%) | |
| Documented | | | | |
| Stage at Dx | | | | <0.001 |
| Stage I | 4 (2.3%) | 1 (1.1%) | 5 (1.9%) | |
| Stage II | 8 (4.5%) | 9 (10.3%) | 17 (6.5%) | |
| Stage III | 29 (16.5%) | 19 (21.8%) | 48 (18.3%) | |
| Stage IV | 122 (69.3%) | 30 (34.5%) | 152 (57.8%) | |
| Unknown/not | 13 (7.4%) | 28 (32.2%) | 41 (15.6%) | |
| documented | | | | |
| Prior Surgery | | | | <0.001 |
| No/unknown | 138 (78.4%) | 49 (56.3%) | 187 (71.1%) | |
| Yes | 38 (21.6%) | 38 (43.7%) | 76 (28.9%) | |
| ECOG | | | | 0.004 |
| 0 | 65 (36.9%) | 16 (18.4%) | 81 (30.8%) | |
| 1 | 69 (39.2%) | 39 (44.8%) | 108 (41.1%) | |
| 2 | 18 (10.2%) | 17 (19.5%) | 35 (13.3%) | |
| 3 | 4 (2.3%) | 7 (8.0%) | 11 (4.2%) | |
| Unknown | 20 (11.4%) | 8 (9.2%) | 28 (10.6%) | |
| Opioid Rx Pre- | | | | 0.828 |
| Therapy | | | | |
| No or unknown | 117 (66.5%) | 59 (67.8%) | 176 (66.9%) | |
| Yes | 59 (33.5%) | 28 (32.2%) | 87 (33.1%) | |
| Albumin | | | | 0.003 |
| Below LLN | 30 (19.5%) | 29 (37.7%) | 59 (25.5%) | |
| Normal | 124 (80.5%) | 48 (62.3%) | 172 (74.5%) | |
| N-Miss | 22 | 10 | 32 | |
| Hemoglobin | | | | 0.486 |
| Below LLN | 114 (71.7%) | 60 (75.9%) | 174 (73.1%) | |
| Normal | 45 (28.3%) | 19 (24.1%) | 64 (26.9%) | |
| N-Miss | 17 | 8 | 25 | |
| AlkPhos | | | | 0.116 |
| Below LLN | 47 (31.5%) | 32 (42.1%) | 79 (35.1%) | |
| Normal | 102 (68.5%) | 44 (57.9%) | 146 (64.9%) | |
| N-Miss | 27 | 11 | 38 | |
| HER2 IHC | | | | 0.552 |
| Equivocal | 2 (1.2%) | 0 (0.0%) | 2 (0.8%) | |
| Negative | 154 (91.1%) | 66 (88.0%) | 220 (90.2%) | |
| Positive | 12 (7.1%) | 8 (10.7%) | 20 (8.2%) | |
| Unknown | 1 (0.6%) | 1 (1.3%) | 2 (0.8%) | |
| N-Miss | 7 | 12 | 19 | |
| TMB | | | | 0.169 |
| Median (Q1, Q3) | 4.3 (2.5, 6.3) | 5.0 (2.5, 8.8) | 4.8 (2.5, 7.5) | |
| MSI by NGS | | | | 0.035 |
| MSI-H | 12 (6.8%) | 13 (14.9%) | 25 (9.5%) | |
| MSS | 164 (93.2%) | 74 (85.1%) | 238 (90.5%) | |
| PD-L1 CPS | | | | 0.165 |
| 0 | 6 (5.5%) | 1 (2.7%) | 7 (4.8%) | |
| 1-5 | 27 (24.5%) | 11 (29.7%) | 38 (25.9%) | |
| 5-10 | 25 (22.7%) | 2 (5.4%) | 27 (18.4%) | |
| ,â•50 | 10 (9.1%) | 4 (10.8%) | 14 (9.5%) | |
| 10-50 | 42 (38.2%) | 19 (51.4%) | 61 (41.5%) | |
| N-Miss | 66 | 50 | 116 | |

TABLE 2
Characteristics of the subgroup of Patient Cohort with TMB of 10 or greater

| | | ICI | | |
|---|---|---|---|---|
| | ICI + Chemo | Monotherapy | | |
| | (N = 24) | (N = 18) | Total (N = 42) | p value |
| Age | | | | 0.602 |
| Median (Q1, Q3) | 70.5 (61.8, 75.0) | 71.0 (61.0, 81.0) | 70.5 (61.0, 78.8) | |
| Sex | | | | 0.71 |
| Female | 8 (33.3%) | 7 (38.9%) | 15 (35.7%) | |
| Male | 16 (66.7%) | 11 (61.1%) | 27 (64.3%) | |
| Race | | | | 0.215 |
| Asian | 0 (0.0%) | 2 (11.1%) | 2 (4.8%) | |
| Black or African | 2 (8.3%) | 0 (0.0%) | 2 (4.8%) | |
| American | | | | |
| Other Race | 3 (12.5%) | 4 (22.2%) | 7 (16.7%) | |
| Unknown/not | 6 (25.0%) | 2 (11.1%) | 8 (19.0%) | |
| documented | | | | |
| White | 13 (54.2%) | 10 (55.6%) | 23 (54.8%) | |
| Practice Type | | | | 0.729 |
| Academic | 2 (8.3%) | 1 (5.6%) | 3 (7.1%) | |
| Community | 22 (91.7%) | 17 (94.4%) | 39 (92.9%) | |
| History of Smoking | | | | 0.094 |
| History of smoking | 18 (75.0%) | 9 (50.0%) | 27 (64.3%) | |
| No history of smoking | 6 (25.0%) | 9 (50.0%) | 15 (35.7%) | |
| H Pylori DNA in | | | | 0.355 |
| Tumor | | | | |
| Not detected | 24 (100.0%) | 18 (100.0%) | 42 (100.0%) | |
| Site of Primary Tumor | | | | 0.482 |
| Esophageal | 8 (33.3%) | 6 (33.3%) | 14 (33.3%) | |
| Gastric | 10 (41.7%) | 10 (55.6%) | 20 (47.6%) | |
| GEJ | 6 (25.0%) | 2 (11.1%) | 8 (19.0%) | |
| Histology | | | | 0.056 |
| Adenocarcinoma | 24 (100.0%) | 13 (72.2%) | 37 (88.1%) | |
| Adenosquamous | 0 (0.0%) | 1 (5.6%) | 1 (2.4%) | |
| Other | 0 (0.0%) | 1 (5.6%) | 1 (2.4%) | |
| Squamous Cell | 0 (0.0%) | 3 (16.7%) | 3 (7.1%) | |
| Carcinoma | | | | |
| Stage at Dx | | | | 0.002 |
| Stage II | 2 (8.3%) | 1 (5.6%) | 3 (7.1%) | |
| Stage III | 5 (20.8%) | 4 (22.2%) | 9 (21.4%) | |
| Stage IV | 17 (70.8%) | 5 (27.8%) | 22 (52.4%) | |
| Unknown/not | 0 (0.0%) | 8 (44.4%) | 8 (19.0%) | |
| documented | | | | |
| Prior Surgery | | | | 0.094 |
| No/unknown | 18 (75.0%) | 9 (50.0%) | 27 (64.3%) | |
| Yes | 6 (25.0%) | 9 (50.0%) | 15 (35.7%) | |
| ECOG | | | | 0.258 |
| 0 | 6 (25.0%) | 1 (5.6%) | 7 (16.7%) | |
| 1 | 11 (45.8%) | 8 (44.4%) | 19 (45.2%) | |
| 2 | 5 (20.8%) | 5 (27.8%) | 10 (23.8%) | |
| 3 | 0 (0.0%) | 2 (11.1%) | 2 (4.8%) | |
| Unknown | 2 (8.3%) | 2 (11.1%) | 4 (9.5%) | |
| Opioid Rx Pre- | | | | 0.7 |
| Therapy | | | | |
| No or unknown | 16 (66.7%) | 13 (72.2%) | 29 (69.0%) | |
| Yes | 8 (33.3%) | 5 (27.8%) | 13 (31.0%) | |
| Albumin | | | | 0.877 |
| Below LLN | 7 (35.0%) | 6 (37.5%) | 13 (36.1%) | |
| Normal | 13 (65.0%) | 10 (62.5%) | 23 (63.9%) | |
| N-Miss | 4 | 2 | 6 | |
| Hemoglobin | | | | 0.739 |
| Below LLN | 14 (70.0%) | 12 (75.0%) | 26 (72.2%) | |
| Normal | 6 (30.0%) | 4 (25.0%) | 10 (27.8%) | |
| N-Miss | 4 | 2 | 6 | |
| AlkPhos | | | | 0.517 |
| Below LLN | 7 (35.0%) | 4 (25.0%) | 11 (30.6%) | |
| Normal | 13 (65.0%) | 12 (75.0%) | 25 (69.4%) | |
| N-Miss | 4 | 2 | 6 | |
| HER2 IHC | | | | 0.189 |
| Negative | 20 (90.9%) | 18 (100.0%) | 38 (95.0%) | |
| Positive | 2 (9.1%) | 0 (0.0%) | 2 (5.0%) | |
| N-Miss | 2 | 0 | 2 | |
| TMB | | | | 0.929 |
| Median (Q1, Q3) | 18.3 (11.9, 40.0) | 19.4 (13.8, 28.4) | 19.4 (12.5, 33.2) | |
| MSI by NGS | | | | 0.327 |
| MSI-H | 11 (45.8%) | 11 (61.1%) | 22 (52.4%) | |
| MSS | 13 (54.2%) | 7 (38.9%) | 20 (47.6%) | |
| PD-L1 CPS | | | | 0.273 |
| 0 | 0 (0.0%) | 1 (10.0%) | 1 (4.5%) | |
| 1-5 | 4 (33.3%) | 3 (30.0%) | 7 (31.8%) | |
| 5-10 | 3 (25.0%) | 0 (0.0%) | 3 (13.6%) | |
| ,â•50 | 1 (8.3%) | 3 (30.0%) | 4 (18.2%) | |
| 10-50 | 4 (33.3%) | 3 (30.0%) | 7 (31.8%) | |
| N-Miss | 12 | 8 | 20 | |

TABLE 3
Lines of therapy for patients who received ICI + chemo

| Line of Therapy | n |
|---|---|
| Fluorouracil, Leucovorin, Nivolumab, Oxaliplatin | 103 |
| FOLFOX, Pembrolizumab | 25 |
| CAPEOX, Pembrolizumab | 8 |
| Capecitabine, Nivolumab, Oxaliplatin | 6 |
| Carboplatin, Paclitaxel, Pembrolizumab | 6 |
| Fluorouracil, Nivolumab, Oxaliplatin | 5 |
| Nivolumab, Oxaliplatin | 3 |
| Capecitabine, Pembrolizumab | 2 |
| Fluorouracil, Irinotecan, Leucovorin, Nivolumab | 2 |
| Capecitabine, Nivolumab, Oxaliplatin, Pembrolizumab | 1 |
| Capecitabine, Oxaliplatin, Paclitaxel, Pembrolizumab | 1 |
| Carboplatin, Nivolumab | 1 |
| Carboplatin, Nivolumab, Paclitaxel | 1 |
| Carboplatin, Oxaliplatin, Paclitaxel, Pembrolizumab | 1 |
| Carboplatin, Paclitaxel Protein-Bound, Pembrolizumab | 1 |
| Fluorouracil, Apalutamide, Leucovorin, Nivolumab, Oxaliplatin, Triptorelin | 1 |
| Fluorouracil, Carboplatin, Leucovorin, Oxaliplatin, Paclitaxel, Pembrolizumab | 1 |
| Fluorouracil, Cisplatin, Leucovorin, Nivolumab | 1 |
| Fluorouracil, Cisplatin, Nivolumab | 1 |
| Fluorouracil, Cisplatin, Pembrolizumab | 1 |
| Fluorouracil, Docetaxel, Leucovorin, Nivolumab, Oxaliplatin | 1 |
| Fluorouracil, Docetaxel, Leucovorin, Oxaliplatin, Pembrolizumab | 1 |
| Fluorouracil, Leucovorin, Pembrolizumab | 1 |
| Fluorouracil, Paclitaxel, Pembrolizumab | 1 |
| Oxaliplatin, Pembrolizumab | 1 |

### Results

A total of 263 patients (176 receiving chemo-ICI and 87 mono-ICI) were included in the study. Patients with TMB<10 mut/mB had more favorable outcomes receiving chemo-ICI vs. mono-ICI for rwPFS (median 5.8 vs. 1.9 months, hazard ratio [HR]: 0.44, 95% confidence interval [CI] 0.26-0.76), p=0.0029) and rwOS (median 11.1 vs. 7.9 months, HR: 0.68, 95% CI 0.40-1.15, p=0.1489). Patients with TMB>10 mut/mB did not have reduced benefit from mono-ICI; curiously less favorable outcomes were observed with chemo-ICI vs. mono-ICI for rwPFS (HR: 3.27, 95% CI 1.19-8.97, p=0.0212) and rwOS (HR: 3.56, 95% CI 1.14-11.08, p=0.0286). Evaluating only the mono-ICI group, the LRT indicated that when TMB was added to a model evaluating only MSI status, there was an improvement in ICI outcome prediction (p=0.006 for rwPFS and p=0.003 for rwOS), but not when MSI was added to TMB (p=0.91 for rwPFS and p=0.40 for rwOS).

### CONCLUSION

aGEJ patients with TMB<10 mut/mB had more favorable outcomes receiving chemo-ICI vs. mono-ICI. In the TMB>10 mut/mB, more favorable outcomes among patients receiving chemo-ICI vs. mono-ICI was not observed. Curiously, more favorable outcomes among those receiving mono-ICI in the TMB>10 mut/mB group was observed. Among patients receiving mono-ICI, TMB better predicted mono-ICI outcomes compared to MSI status alone.

What is claimed is:

1. A method for treating an individual having a gastroesophageal cancer, the method comprising:
(a) determining a tumor mutational burden (TMB) score for a biopsy sample obtained from the individual; and
(b) administering an immune checkpoint inhibitor without a chemotherapy regimen to the individual if the TMB score is at or higher than a threshold TMB score.

2. A method for identifying an individual having a gastroesophageal cancer for treatment with an immune checkpoint inhibitor therapy without a chemotherapy regimen comprising determining a tumor mutational burden (TMB) score for a biopsy sample obtained from the individual, wherein if the TMB score is at or higher than a threshold TMB score the individual is identified for treatment with an immune checkpoint inhibitor therapy without a chemotherapy regimen.

3. A method of identifying one or more treatment options for an individual having a gastroesophageal cancer, the method comprising:
(a) determining a tumor mutational burden (TMB) score for a biopsy sample obtained from the individual; and
(b) generating a report comprising one or more treatment options identified for the individual, wherein a TMB score that is at or higher than a threshold TMB score identifies the individual as one who may benefit from treatment with an immune checkpoint inhibitor therapy without a chemotherapy regimen.

4. The method of claim 1, wherein the threshold TMB score is about 8 mutations/Mb, about 9 mutations/Mb, about 10 mutations/Mb, about 11 mutations/Mb, about 12 mutations/Mb, about 13 mutations/Mb, about 14 mutations/Mb, about 15 mutations/Mb, about 16 mutations/Mb, about 17 mutations/Mb, about 18 mutations/Mb, about 19 mutations/Mb, or about 20 mutations/Mb.

5. The method of claim 4, wherein the threshold TMB score is about 10 mutations/Mb.

6. The method of claim 1, wherein the TMB score is determined based on between about 100 kb to about 10 Mb of sequenced DNA.

7. The method of claim 6, wherein the TMB score is determined based on between about 0.8 Mb to about 1.1 Mb of sequenced DNA.

8. The method of claim 2, further comprising administering an immune checkpoint inhibitor without a chemotherapy regimen to the individual if the TMB score is at or higher than the threshold TMB score.

9. The method of claim 1, further comprising administering an immune checkpoint inhibitor and a chemotherapy regimen to the individual if the TMB score is less than the threshold TMB score.

10. The method of claim 1, wherein the immune checkpoint inhibitor therapy is a first line therapy for the gastroesophageal cancer.

11. The method of claim 1, wherein the immune checkpoint inhibitor comprises a small molecule inhibitor, an antibody, a nucleic acid, an antibody-drug conjugate, a recombinant protein, a fusion protein, a natural compound, a peptide, a PROteolysis-TArgeting Chimera (PROTAC), a cellular therapy, a treatment for cancer being tested in a clinical trial, an immunotherapy, or any combination thereof.

12. The method of claim 11, wherein the immune checkpoint inhibitor is a PD-1 inhibitor.

13. The method of claim 12, wherein the PD-1 inhibitor comprises one or more of nivolumab, pembrolizumab, cemiplimab, or dostarlimab.

14. The method of claim 11, wherein the immune checkpoint inhibitor is a PD-L1 inhibitor.

15. The method of claim 14, wherein the PD-L1 inhibitor comprises one or more of atezolizumab, avelumab, or durvalumab.

16. The method of claim 11, wherein the immune checkpoint inhibitor is a CTLA-4 inhibitor.

17. The method of claim 16, wherein the CTLA-4 inhibitor comprises ipilimumab.

18. The method of claim 1, wherein the immune checkpoint inhibitor therapy is a single-active-agent therapy.

19. The method of claim 1, wherein the immune checkpoint inhibitor therapy comprises two or more active agents.

20. The method of claim 1, wherein the immune checkpoint inhibitor therapy comprises a first round of an immune checkpoint inhibitor and a subsequent round of therapy with a different immune checkpoint inhibitor.
